SEQUENCE LISTING (49 sequences)

SEQ ID NO: 1 - protein, 20 aa, Artificial Sequence
  p52
TSFFTWFMVIALLGVWTSVA

SEQ ID NO: 2 - protein, 15 aa, Artificial Sequence
  p103
AKVLLGLKERSTSEP

SEQ ID NO: 3 - protein, 19 aa, Artificial Sequence
  p148
KEQIQSLLHEMVHAEHVEG

SEQ ID NO: 4 - protein, 16 aa, Artificial Sequence
  p322
QKAKVKKKKPKLLNKF

SEQ ID NO: 5 - protein, 18 aa, Artificial Sequence
  p415
PADLLKLSLKRRSDRQQF

SEQ ID NO: 6 - protein, 18 aa, Artificial Sequence
  p427
SDRQQFLGHMRGSLLTLQ

SEQ ID NO: 7 - protein, 16 aa, Artificial Sequence
  p437
RGSLLTLQRLVQLFPN

SEQ ID NO: 8 - protein, 16 aa, Artificial Sequence
  p443
LQRLVQLFPNDTSLKN

SEQ ID NO: 9 - protein, 18 aa, Artificial Sequence
  p492
VHYGFILKAQNKIAESIP

SEQ ID NO: 10 - protein, 20 aa, Artificial Sequence
  p557
ASVWQRSLYNVNGLKAQPWW

SEQ ID NO: 11 - protein, 17 aa, Artificial Sequence
  p58
TGYTELVKSLERNWKLI

SEQ ID NO: 12 - protein, 20 aa, Artificial Sequence
  p588
KSLERNWKLIRDEGLAVMDK

SEQ ID NO: 13 - protein, 14 aa, Artificial Sequence
  p725
HEVWQDASSFRLIF

SEQ ID NO: 14 - protein, 17 aa, Artificial Sequence
  p731
ASSFRLIFIVDVWHPEL

SEQ ID NO: 15 - protein, 19 aa, Artificial Sequence
  [sequence omitted]
VDVWHPELTPQQRRSLPAI

SEQ ID NO: 16 - protein, 9 aa, Artificial Sequence
  ASPH48
GLSGTSFFT

SEQ ID NO: 17 - protein, 9 aa, Artificial Sequence
  ASPH53
SFFTWFMVI

SEQ ID NO: 18 - protein, 9 aa, Artificial Sequence
  ASPH58
FMVIALLGV

SEQ ID NO: 19 - protein, 9 aa, Artificial Sequence
  ASPH62
ALLGVWTSV

SEQ ID NO: 20 - protein, 9 aa, Artificial Sequence
  ASPH72
VVWFDLVDY

SEQ ID NO: 21 - protein, 9 aa, Artificial Sequence
  ASPH79
DYEEVLGKL

SEQ ID NO: 22 - protein, 9 aa, Artificial Sequence
  ASPH81
EEVLGKLGI

SEQ ID NO: 23 - protein, 9 aa, Artificial Sequence
  ASPH252
TDDVTYQVY

SEQ ID NO: 24 - protein, 9 aa, Artificial Sequence
  ASPH258
QVYEEQAVY

SEQ ID NO: 25 - protein, 9 aa, Artificial Sequence
  ASPH261
EEQAVYEPL

SEQ ID NO: 26 - protein, 9 aa, Artificial Sequence
  ASPH371
YPQSPRARY

SEQ ID NO: 27 - protein, 9 aa, Artificial Sequence
  ASPH374
SPRARYGKA

SEQ ID NO: 28 - protein, 9 aa, Artificial Sequence
  ASPH406
QEVASLPDV

SEQ ID NO: 29 - protein, 9 aa, Artificial Sequence
  ASPH411
LPDVPADLL

SEQ ID NO: 30 - protein, 9 aa, Artificial Sequence
  ASPH475
KVYEEVLSV

SEQ ID NO: 31 - protein, 9 aa, Artificial Sequence
  ASPH478
EEVLSVTPN

SEQ ID NO: 32 - protein, 9 aa, Artificial Sequence
  ASPH484
TPNDGFAKV

SEQ ID NO: 33 - protein, 9 aa, Artificial Sequence
  ASPH488
GFAKVHYGF

SEQ ID NO: 34 - protein, 9 aa, Artificial Sequence
  ASPH491
KVHYGFILK

SEQ ID NO: 35 - protein, 9 aa, Artificial Sequence
  ASPH503
KIAESIPYL

SEQ ID NO: 36 - protein, 9 aa, Artificial Sequence
  ASPH521
GTDDGRFYF

SEQ ID NO: 37 - protein, 9 aa, Artificial Sequence
  ASPH537
RVGNKEAYK

SEQ ID NO: 38 - protein, 9 aa, Artificial Sequence
  ASPH557
ASVWQRSLY

SEQ ID NO: 39 - protein, 9 aa, Artificial Sequence
  ASPH563
SLYNVNGLK

SEQ ID NO: 40 - protein, 9 aa, Artificial Sequence
  ASPH582
GYTELVKSL

SEQ ID NO: 41 - protein, 9 aa, Artificial Sequence
  ASPH611
LFLPEDENL

SEQ ID NO: 42 - protein, 9 aa, Artificial Sequence
  ASPH681
GPTNCRLRM

SEQ ID NO: 43 - protein, 9 aa, Artificial Sequence
  ASPH693
LVIPKEGCK

SEQ ID NO: 44 - protein, 9 aa, Artificial Sequence
  ASPH701
KIRCANETR

SEQ ID NO: 45 - protein, 9 aa, Artificial Sequence
  ASPH711
WEEGKVLIF

SEQ ID NO: 46 - protein, 758 aa, Homo sapiens [sequence omitted]

SEQ ID NO: 47 - protein, 253 aa, Artificial Sequence
  the first third of the human ASPH amino acid sequence
MAQRKNAKSSGNSSSSGSGSGSTSAGSSSPGARRETKHGGHKNGRKGGLSGTSFFTWFMVIALLGVWTSVAVVWFDLVDYEEVLGKLGIYDADGDGDFDVDDAKVLLGLKERSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTD

SEQ ID NO: 48 - protein, 253 aa, Artificial Sequence
  the last third of the human ASPH amino acid sequence
ESIPYLKEGIESGDPGTDDGRFYFHLGDAMQRVGNKEAYKWYELGHKRGHFASVWQRSLYNVNGLKAQPWWTPKETGYTELVKSLERNWKLIRDEGLAVMDKAKGLFLPEDENLREKGDWSQFTLWQQGRRNENACKGAPKTCTLLEKFPETTGCRRGQIKYSIMHPGTHVWPHTGPTNCRLRMHLGLVIPKEGCKIRCANETRTWEEGKVLIFDDSFEHEVWQDASSFRLIFIVDVWHPELTPQQRRSLPAI

SEQ ID NO: 49 - DNA, 2324 bp, Homo sapiens
cggaccgtgcaatggcccagcgtaagaatgccaagagcagcggcaacagcagcagcagcggctccggcagcggtagcacgagtgcgggcagcagcagccccggggcccggagagagacaaagcatggaggacacaagaatgggaggaaaggcggactctcgggaacttcattcttcacgtggtttatggtgattgcattgctgggcgtctggacatctgtagctgtcgtttggtttgatcttgttgactatgaggaagttctaggaaaactaggaatctatgatgctgatggtgatggagattttgatgtggatgatgccaaagttttattaggacttaaagagagatctacttcagagccagcagtcccgccagaagaggctgagccacacactgagcccgaggagcaggttcctgtggaggcagaaccccagaatatcgaagatgaagcaaaagaacaaattcagtcccttctccatgaaatggtacacgcagaacatgttgagggagaagacttgcaacaagaagatggacccacaggagaaccacaacaagaggatgatgagtttcttatggcgactgatgtagatgatagatttgagaccctggaacctgaagtatctcatgaagaaaccgagcatagttaccacgtggaagagacagtttcacaagactgtaatcaggatatggaagagatgatgtctgagcaggaaaatccagattccagtgaaccagtagtagaagatgaaagattgcaccatgatacagatgatgtaacataccaagtctatgaggaacaagcagtatatgaacctctagaaaatgaagggatagaaatcacagaagtaactgctccccctgaggataatcctgtagaagattcacaggtaattgtagaagaagtaagcatttttcctgtggaagaacagcaggaagtaccaccagaaacaaatagaaaaacagatgatccagaacaaaaagcaaaagttaagaaaaagaagcctaaacttttaaataaatttgataagactattaaagctgaacttgatgctgcagaaaaactccgtaaaaggggaaaaattgaggaagcagtgaatgcatttaaagaactagtacgcaaataccctcagagtccacgagcaagatatgggaaggcgcagtgtgaggatgatttggctgagaagaggagaagtaatgaggtgctacgtggagccatcgagacctaccaagaggtggccagcctacctgatgtccctgcagacctgctgaagctgagtttgaagcgtcgctcagacaggcaacaatttctaggtcatatgagaggttccctgcttaccctgcagagattagttcaactatttcccaatgatacttccttaaaaaatgaccttggcgtgggatacctcttgataggagataatgacaatgcaaagaaagtttatgaagaggtgctgagtgtgacacctaatgatggctttgctaaagtccattatggcttcatcctgaaggcacagaacaaaattgctgagagcatcccatatttaaaggaaggaatagaatccggagatcctggcactgatgatgggagattttatttccacctgggggatgccatgcagagggttgggaacaaagaggcatataagtggtatgagcttgggcacaagagaggacactttgcatctgtctggcaacgctcactctacaatgtgaatggactgaaagcacagccttggtggaccccaaaagaaacgggctacacagagttagtaaagtctttagaaagaaactggaagttaatccgagatgaaggccttgcagtgatggataaagccaaaggtctcttcctgcctgaggatgaaaacctgagggaaaaaggggactggagccagttcacgctgtggcagcaaggaagaagaaatgaaaatgcctgcaaaggagctcctaaaacctgtaccttactagaaaagttccccgagacaacaggatgcagaagaggacagatcaaatattccatcatgcaccccgggactcacgtgtggccgcacacagggcccacaaactgcaggctccgaatgcacctgggcttggtgattcccaaggaaggctgcaagattcgatgtgccaacgagaccaggacctgggaggaaggcaaggtgctcatctttgatgactcctttgagcacgaggtatggcaggatgcctcatctttccggctgatattcatcgtggatgtgtggcatccggaactgacaccacagcagagacgcagccttccagcaatttagcatgaattcatgcaagcttgggaaactctggagaga

(19) United States
(12) Patent Application Publication
(10) Pub. No.: US 2022/0054614 A1
(43) Pub. Date: Feb. 24, 2022

(54) INHIBITION OF ASPH EXPRESSING TUMOR GROWTH AND PROGRESSION

(71) Applicant: Rhode Island Hospital, Providence, RI (US)

(72) Inventors: Jack R. Wands, Warwick, RI (US); Xiaoqun Dong, Warwick, RI (US)

(21) Appl. No.: 17/413,913

(22) PCT Filed: Dec. 13, 2019
(86) PCT No.: PCT/US19/66174

§ 371 Date: Jun. 14, 2021

Related U.S. Application Data
(60) Provisional application No. 62/779,422, filed on Dec. 13, 2018.

(51) Int. Cl. A61K 39/00; A61K 39/395; A61P 35/00

(52) U.S. Cl. CPC A61K 39/001154; A61K 39/3955; A61K 2039/545; A61K 2039/5154; A61K 2039/5256; A61P 35/00

(57) ABSTRACT

Disclosed are compositions and methods for an immunotherapy in a subject containing a vaccine construct for an immunization against a purified tumor antigen and a checkpoint inhibitor for treating a tumor in the subject, in which the tumor is characterized as comprising a low frequency of neoantigen expression and the composition potentiates an anti-tumor immune response without inducing autoimmunity in the subject. A pharmaceutical composition containing the composition as an active component and a pharmaceutically acceptable carrier, and a combinatorial composition containing a vaccine construct for an immunization against a purified tumor antigen and an immune checkpoint inhibitor, in which the tumor is characterized as comprising a low frequency of neoantigen expression, are also described.

### RELATED APPLICATIONS

This application claims the benefit of priority under 35 U.S.C. § 119(e) to U.S. Provisional Application No. 62/779,422, filed Dec. 13, 2018, the entire contents of which is incorporated herein by reference in its entirety.

### INCORPORATION BY REFERENCE OF SEQUENCE LISTING

The contents of the sequence listing text file named “21486-642001WO_Sequence_Listing_ST25.txt”, which was created on Nov. 11, 2019 and is 24,576 bytes in size, is hereby incorporated by reference in its entirety.

### FIELD OF THE INVENTION

The present invention relates to immunotherapies for treating cancer.

### BACKGROUND

Aspartyl asparaginyl β-hydroxylase (ASPH), a transmembrane oncofetal protein and tumor associated antigen (TAA), presents on many types of maligant cells but not normal cells in adult (except for placenta). Approximately 80% of solid tumors overexpress ASPH (compared to adjacent normal tissue), an oncogene required for proliferation, survival, migration, invasion, stemness, and metastasis of tumor cells. Although significant progress has been made in the field of cancer therapy, there are few effective approaches currently available for these devastating diseases.

### SUMMARY OF THE INVENTION

The invention provides a solution to the longstanding problem of cancer therapy by providing a method for achieving unanticipated and dramatic inhibition of tumor development, growth, relapse and progression as well as metastatic spread to other sites and organs in the body. The antigen specific immune response to specifically defined purified tumor antigen(s) (e.g., a lambda phage 1 expressing N terminal peptides of ASPH (SEQ ID NO: 47 in Table 4)) of a specific class of tumor characterized by a relatively low tumor mutation burden (TMB) (e.g., carrying 0.001 to ≤1 somatic mutation/megabase, compared to >1, 10, 100 or >100 somatic mutations/megabase, which is considered as relatively “high” when appropriate) or a relatively low frequency of neoantigen expression can be greatly amplified with the sequential or concurrent administration of immune modulators (including checkpoint inhibitors). For example, low TMB is relative to high TMB, e.g., 0.001 to ≤1 somatic mutation/megabase (low TMB) as compared to >1, 10, 100 or >100 somatic mutations/megabase (high TMB).

Administration is meant to include concurrent or sequential administration of a compound or composition individually or in combination (more than one compound or agent). For example, the vaccine construct for an immunization against a purified tumor antigen may be administered concurrent with the checkpoint inhibitor.

In other examples, the vaccine construct for an immunization against a purified tumor antigen may be administered sequential to the checkpoint inhibitor. By “sequential” administration is meant a time difference of from seconds, minutes, hours or days between the administration of the two types of agents (e.g., the vaccine construct for an immunization against a purified tumor antigen and checkpoint inhibitor). These agents may be administered in any order.

This invention has widespread application for the treatment of hematologic malignancies (such as lukemia) and various solid tumors, such as malignancies originated from liver, pancreas, stomach, esophagus, colon, rectum, bile duct, gallbladder, soft tissue (e.g. sarcomas), central nervous system (e.g., glioblastoma multiforme), head and neck (e.g., squamous cell), bone (osteosarcoma), cartilage (chondrosarcoma), lung (e.g., non-small cell), urinary & genital tract (e.g., kidney, ovary, cervix), prostate and breast (including triple negative). In some embodiments, the methods do not comprise treatment of a class of tumors characterized by a relatively high frequency of tumor-specific DNA alterations or a relatively high TMB (e.g., carrying >100 somatic mutation/megabase, compared to 1 somatic mutation/megabase, which is considered as relatively “low” when appropirate) that leads to generation of neoantigens, such as melanoma and small cell lung cancer.

These methods stimulate immune responses to a single chemically defined transmembrane antigen, e.g., ASPH, that is overexpressed in a majority of human solid tumors. Subsequently, antigen specific B and T cell immune responses are generated with various vaccine modalities, e.g., phage, dendritic cells, DNA-based, RNA-based, extrachromosomal DNA (ecDNA)-based, and peptide-based formulations, with surprising levels of amplification by immune modulators (including checkpoint inhibitors). Advantages of the methods described herein include very few or no adverse side effects and use of a significantly reduced amount of immune checkpoint (e.g., PD-1, PD-L1) inhibitors due to precise targeting of a specific and well-defined antigenic sequence, e.g., full-length or alternative splicing variants of ASPH, e.g., N-terminal and/or C-terminal epitopes of ASPH.

Accordingly, the invention features a composition and methods for immunotherapy in a subject comprising concurrently or sequentially a vaccine construct for an immunization against a purified tumor associated antigen (and its derivatives) and an immune modulator (such as a checkpoint inhibitor) for treating tumors in the subject, wherein the composition potentiates an antigen-specific anti-tumor adaptive immune response without inducing autoimmunity in the subject. Preferably, the tumor is characterized as comprising a low frequency of neoantigen expression. For instance, Yarchoan et al. and Schumacher and Schreiber described quantifying a relatively low frequency of TMB to create neoantigens like that found in pancreatic cancer and hepatocellular carcinoma (HCC) (see, e.g., Yarchoan et al., Nat. Rev. Cancer. 2017 April; 17(4):209-222. Epub 2017 Feb. 24; Schumacher and Schreiber, Science 2015 Apr. 3; 348(6230):69-74, the entire contents of which are hereby incorporated by reference). Yet, both pancreatic cancer and HCC have very high levels of ASPH expression on tumor cells but not normal cells. In addition, a relatively high frequency of neoantigen generation is found in non-small cell lung cancer, which also highly expresses ASPH. So, there is little relationship of ASPH expression with neoantigen generation or TMB in most solid tumors as described in Table 1 below.

For example, the purified, e.g., a single chemically defined antigen, is aspartate beta-hydroxylase (ASPH) or an antigen fragment and their derivatives (e.g., alternative splicing variants, truncated, mutant, fusion or post-translational modification) thereof. For example, the vaccine construct expresses a purified ASPH antigen and its derivatives. Purified ASPH antigen and its derivatives comprises e.g., the mature full-length antigen (SEQ ID NO: 46) as well as a purified N-terminal ASPH peptide, preferably, the first third of the ASPH protein sequence (e.g., SEQ ID NO: 47), or a purified C-terminal ASPH peptide, preferably, the last third of the ASPH peptide sequence (e.g., SEQ ID NO: 48). Exemplary antigens include a purified peptide selected from the group consisting of SEQ ID NOs: 1-45, e.g., a human leukocyte antigen (HLA) class II restricted sequence of TGYTELVKSLERNWKLI (SEQ ID NO: 11) or an HLA class I restricted sequence of YPQSPRARY (SEQ ID NO: 26).

In some embodiments, the vaccine construct comprises a phage vaccine or a dendritic cell vaccine. For example, the phage vaccine is a lambda phage-based vaccine and wherein the dendritic cell vaccine comprises isolated ASPH (and its derivatives)-loaded (e.g., incubated, transfected) dendritic cells.

The composition and methods also encompass an immune modulator (including a checkpoint inhibitor), e.g., to implement Programmed cell death protein-1 (PD-1) signal blockade or inhibition, e.g., PD-1 signal blockade encompasses a PD-1 inhibitory antibody, a PD-1 inhibitory nucleic acid, a PD-1 inhibitory small molecule or a PD-1 ligand mimetic. In some examples, PD-1 signal blockade is implemented using an anti-PD-1 monoclonal antibody or an anti-Programmed death-ligand 1 (PD-L1) monoclonal antibody or anti-Programmed death-ligand 2 (PD-L2) monoclonal antibody.

The composition reduces tumor development, growth, relapse/recurrence, progression, or metastatic spread to a different site/organ or a combination thereof. The composition also stimulates an endogenous adaptive (cellular and humoral) immune system. For example, the composition stimulates generation of an ASPH-specific B cell immune response, generation of an ASPH-specific T cell immune response, or generation of a combination thereof and/or stimulates activation of a cluster of differentiation 8 (CD8)⁺ cell, activation of a cluster of differentiation 4 (CD4)⁺ cell, activation of matured dendritic cell, or activation of a combination thereof.

As discussed above, the tumor is a cancer with a relatively low TMB or a relatively low neoantigen burden. For example, the frequency of mutations in ASPH to generate neoantigens is relatively low, i.e., infrequent (e.g., carrying 0.001 to ≤1 somatic mutation/megabase). TMB in a sample from a test subject is compared to TMB in a reference sample of a cell or cells of known cancer status. The threshold for determining whether a test sample is scored positive can be altered depending on the sensitivity or specificity desired.

As used herein the term, “neoantigen” is an antigen encoded by tumor-specific mutated genes that has at least one alteration that makes it distinct from the corresponding wild-type, parental antigen. Tumor neoantigen, belonging to tumor-specific antigen (TSA), is the repertoire of peptides being displayed on the surface of tumor cells and specifically recognized by neoantigen-specific T cell receptors (TCRs) in the context of major histocompatibility (MHCs) complexes. For example, the antigen is a protein and a neoantigen is one that occurs via mutations in a tumor cell or post-translational modifications specific to a tumor cell. A neoantigen can include a polypeptide sequence or a nucleotide sequence. A mutation can include a frameshift or non-frameshift indel, missense or nonsense substitution, splice site alteration, genomic rearrangement or gene fusion, structural variants, or any genomic or expression alteration giving rise to a neoORF. A mutations can also include a splice variant (such as exon skipping) caused by alternative splicing. As used herein the term “tumor neoantigen” is a neoantigen present in a subject's tumor cell or tissue but not in the subject's corresponding normal cell or tissue. In embodiments, as used herein the term “neoantigen-based vaccine” is a vaccine construct based on one or more neoantigens, e.g., a plurality of neoantigens.

Also, within the invention is an immunotherapeutic method of treating a tumor in a subject, comprising currently or sequentially administering to the subject a vaccine construct for an immunization against a purified tumor antigen, the tumor being characterized as comprising a relatively low frequency of neoantigen expression or a relatively low frequency of TMB, and an immune modulator (including a checkpoint inhibitor). For example, the immune checkpoint inhibitor (e.g., inhibitor of PD-1, PD-L1, or PD-L2, as described above) is administered together with, e.g., concurrently, before or after, e.g., sequentially, administration of the tumor antigen vaccine (phage vaccine, dendritic cell vaccine, or other vaccine formulation containing the subject's antigen, e.g., purified ASPH or antigenic fragments or their derivatives thereof). The method potentiates an anti-tumor immune response without inducing autoimmunity in subject. For example, vaccine construct expresses a purified ASPH antigen such as a purified N-terminal ASPH peptide or a purified C-terminal ASPH peptide. Exemplary peptides are described above and sequences provided in Table 4 below.

The method encompasses prophylactic immunization as well as one or more booster immunization (s). For example, the prophylactic immunization comprises administering the vaccine construct to the subject three times spaced one week apart. The booster immunization comprises administering the vaccine construct to the subject three times spaced one week apart. The immune checkpoint inhibitor is administered concurrently or subsequently with the vaccine construct, e.g., the checkpoint inhibitor is administered twice per week for 5 or 6 weeks. Moreover, afterwards, a long-term booster may also include an immunization once per 3 months, 6 months, 12 months, 24 months, 36 months, 48 months and thereof.

The class of tumor to be treated is described above and is preferably a solid tumor such as hepato cellular carcinoma (HCC), cholangiocarcinoma, non-small cell lung cancer, (triple negative) breast cancer, gastric cancer, pancreatic cancer, esophageal cancer, gallbladder cancer, soft tissue sarcomas (such as liposarcoma), osteosarcoma, chondrosarcoma, colon and rectal cancer, renal cancer, head and neck squamous cell carcinoma, myeloid or lymphoid leukemia, urinary and genital tract (such as cervial) cancer, ovary cancer, thyroid cancer, prostate cancer, head and neck cancer, and glioblastoma multiforme. For example, the tumor is an HCC.

The method is associated with reducing tumor development, growth, recurrence/relapse, progression, or metastatic spread to a different site/organ, or a combination thereof. For example, the method achieves the aforementioned anti-tumor effects by stimulating an endogenous adaptive (cellular and humoral) immune system, e.g., via generation of an ASPH-specific B cell immune response, generation of an ASPH-specific T cell immune response, or generation of a combination thereof. More specifically, the method is associated with activation of a CD8⁺ cell, activation of a CD4⁺ cell, activation of matured dendritic cell, or activation of a combination thereof.

Also within the invention is a pharmaceutical composition for immunotherapy in a subject comprising a vaccine construct for an immunization against a purified tumor antigen and an immune modulator (such as a checkpoint inhibitor) for treating a tumor in the subject, wherein the composition potentiates an anti-tumor immune response without inducing autoimmunity in the subject as an active component, and a pharmaceutically acceptable carrier.

Another aspect of the invention includes a combinatorial composition comprising concurrently or sequentially a vaccine construct for an immunization against a purified tumor antigen, and an immune checkpoint inhibitor. Preferably, the tumor is characterized as comprising preferably a relatively low frequency of TMB or neoantigen expression. The purified tumor antigen, e.g., a single chemically defined antigen, is, for example, an aspartate beta-hydroxylase (ASPH) or an antigen fragment and their derivatives thereof. For example, the vaccine construct expresses a purified ASPH antigen, which comprises the mature full-length antigen, a purified N-terminal ASPH peptide or a purified C-terminal ASPH peptide. Examples of purified ASPH antigens include a purified peptide selected from the group consisting of SEQ ID NOs: 1-45, for example, a human leukocyte antigen (HLA) class II restricted sequence of TGYTELVKSLERNWKLI (SEQ ID NO: 11) or an HLA class I restricted sequence of YPQSPRARY (SEQ ID NO:26).

Immune checkpoints include co-stimulatory and inhibitory elements intrinsic to a subject's immune system Immune checkpoints aid in maintaining self-tolerance and modulating the duration and amplitude of physiological immune responses to prevent injury to tissues when a subject's immune system responds to pathogenic infection. An immune response can also be initiated when a T-cell recognizes “foreign” antigens that are unique to a tumor cell (e.g. non-self-antigens or tumor neo-antigens) or are characteristics of a tumor cell (e.g. tumor-associated antigens (TAAs)). The equilibrium between the co-stimulatory and inhibitory signals used to control a subject's immune response from T-cells can be modulated by immune checkpoints and their derivatives. After T-cells mature and activate in the thymus, T-cells can travel to sites of inflammation and injury/damage to perform defense functions. T-cell function can occur either via direct action or through the recruitment of cytokines and membrane ligands involved in defensive immune system. The steps involved in T-cell maturation, activation, proliferation, and function can be regulated through co-stimulatory and inhibitory signals, namely through immune checkpoints. Tumors can dysregulate, reprogram or edit checkpoint function as an immune-escape mechanism. Thus, the development of modulators of immune checkpoints can have therapeutic value. Non-limiting examples of immune checkpoint molecules and their derivatives (e.g., post-translational modifications, truncated forms, fusion proteins) include Lymphocyte-activation gene 3 (LAG3), glucocorticoid-induced TNFR-related protein (GITR), B- and T-lymphocyte attenuator (BTLA), killer immunoglobulin-like receptor (KIR), V-domain Ig suppressor of T cell activation (VISTA) (VISTA), cytotoxic T-lymphocyte antigen 4 (CTLA4; also known as CD152 (Cluster of differentiation 152), B7-H3 (CD276), V-set domain-containing T-cell activation inhibitor 1 (VTCN1)/B7-H4, B and T Lymphocyte Attenuator (BTLA)/CD272, OX40/CD134, CD27, CD70, CD137, CD122, CD180, Thymocyte selection-associated high mobility group box protein (TOX), CD28, Inducible T-cell Co-Stimulator (ICOS), T-cell immunoglobulin and mucin domain-containing protein 3 (TIM3 also known as Hepatitis A virus cellular receptor 2 (HAVCR2)), T cell immunoreceptor with Ig and ITIM domains (TIGIT), Indoleamine 2,3-dioxygenase (IDO), NADPH oxidase 2 (NOX2), Sialic acid-binding immunoglobulin-type lectin 7 (SIGLEC7)/CD328, SIGLEC9/CD329, SIGLECT-15, adenosine receptor 2 (A2aR), programmed death protein (PD1), programmed death protein ligand 1 (PD-L1), programmed death protein 2 (PD-2), programmed death protein ligand 2 (PD-L2)/B7-DC, CD40, and CD40 ligand (CD40L)/CD154. In embodiments, the immune checkpoint inhibitor comprises e.g., PD-1. In other embodiments, the immune checkpoint inhibitor comprises e.g., PD-L1.

An immune checkpoint inhibitor is a compound or composition that specifically binds to an immune checkpoint protein. For example, the inhibitor comprises a protein polypeptide or a non-protein compound, including for example a small molecule. For example, the immune checkpoint protein comprises such as LAG3, BTLA, KIR, CTLA4, ICOS, TIM3, A2aR, PD1, PD-L1, PD-L2, and CD40L. In some embodiments, the polypeptide or protein is an antibody or antigen-binding fragment thereof. In some embodiments, the immune checkpoint inhibitor is an interfering nucleic acid molecule. In some embodiments, the interfering nucleic acid molecule is an siRNA molecule, an shRNA molecule or an antisense RNA molecule. In some embodiments, the immune checkpoint inhibitor comprises of Opdivo/nivolumab, Keytruda/pembrolizumab, Tecentriq/Atezolizumab (anti-PD-L1 mAb), Bavencio/Avelumab (anti-PD-L1 mAb), Imfinzi/Durvalumab (anti-PD-L1 mAb), Libtayo/Cemiplimab-rwlc (anti-PD-1 mAb), pidilizumab, CA-170 (PD-L1/VISTA antagonist), CA-327 (PD-L1/TIM3 antagonist), AMP-224, AMP-514, STI-A1110, TSR-042, RG-7446, BMS-936559, BMS-936558, MK-3475, CT 011, MPDL3280A, MEDI-4736, MSB-0020718C, AUR-012 and STI-A1010.

By, “small molecule” may be referred to broadly as an organic, inorganic or organometallic compound with a low molecular weight compound (e.g., a molecular weight of less than about 2,000 Da or less than about 1,000 Da). The small molecule may have a molecular weight of less than about 2,000 Da, a molecular weight of less than about 1,500 Da, a molecular weight of less than about 1,000 Da, a molecular weight of less than about 900 Da, a molecular weight of less than about 800 Da, a molecular weight of less than about 700 Da, a molecular weight of less than about 600 Da, a molecular weight of less than about 500 Da, a molecular weight of less than about 400 Da, a molecular weight of less than about 300 Da, a molecular weight of less than about 200 Da, a molecular weight of less than about 100 Da, or a molecular weight of less than about 50 Da.

Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecules can be synthetically engineered to consist of a fragment, or small portion, or a longer amino acid chain to fill a binding pocket of an enzyme. Typically, small molecules are less than one kilodalton.

For example, the vaccine construct comprises a phage vaccine or a dendritic cell vaccine. Exemplary phage vaccines include a lambda phage-based vaccine, and exemplary dendritic cell vaccines include isolated ASPH-loaded dendritic cells.

For another example, the immune checkpoint inhibitor is a PD-1 blockade or inhibition, such as a PD-1 inhibitory antibody, a PD-1 inhibitory nucleic acid, a PD-1 inhibitory small molecule or a PD-1 ligand mimetic. In some embodiments, the PD-1 signal blockade is an anti-PD-1 monoclonal antibody or an anti-PD-L1 monoclonal antibody.

In aspects, provided herein is an immunotherapeutic method of inhibiting metastasis in a subject, comprising: administering to the subject a vaccine construct for an immunization against a purified tumor antigen, and an immune checkpoint inhibitor. For example, the the vaccine is administered through intradermal, subcutaneous, intranasal, intramuscular, intratumoral, intranodal, intralymphatic, intravenous, intragastric, intraperitoneal, intravaginal, intravesical, percutaneous, or other routes.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, for example, the liver, which site is referred to as a primary tumor, e.g., primary liver cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations (e.g., liver, bone, brain) spread from a primary tumor originated in other organs, e.g., breast.

In other aspects, provided herein is an immunotherapeutic method of inhibiting growth of a primary tumor in a subject, comprising: concurrently or sequentially administering to the subject a vaccine construct for an immunization against a purified tumor antigen, and an immune modulator. In embodiments, the immune modulator is a checkpoint inhibitor. For example, an immunotherapeutic method of inhibiting growth of a primary tumor in a subject, comprising (e.g., using a protocol as shown in FIG. 1 or FIG. 9), concurrently and/or sequentially administering to the subject a vaccine construct for an immunization against a purified tumor antigen, and an immune modulator (including a checkpoint inhibitor).

Administration is meant to include concurrent or sequential administration of a compound or composition individually or in combination (more than one compound or agent). For example, the vaccine construct for an immunization against a purified tumor antigen may be administered concurrent with the checkpoint inhibitor.

In other examples, the vaccine construct for an immunization against a purified tumor antigen may be administered sequential to the checkpoint inhibitor. By “sequential” administration is meant a time difference of from seconds, minutes, hours or days between the administration of the two types of agents (e.g., the vaccine construct for an immunization against a purified tumor antigen and checkpoint inhibitor). These agents may be administered in any order.

The compositions and methods described confer a beneficial therapeutic effect on subjects diagnosed with and suffering from a cancer/malignant tumor growth in that the therapeutic method leads to a synergistic inhibition of tumor growth or tumor metastases in the subject.

Each embodiment disclosed herein is contemplated as being applicable to each of the other disclosed embodiments. Thus, all combinations of the various elements described herein are within the scope of the invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### DESCRIPTION OF THE DRAWINGS

FIG. 1A depicts a diagram of an experimental protocol for a murine model of liver cancer using aspartyl asparaginyl β-hydroxylase (ASPH)-expressing BNL (e.g., liver; BNL 1ME A.7R.1 cell line (ATCC Accession No. TIB-75)) cells.

FIG. 1B depicts a schematic of an immunization protocol.

FIG. 2A is an image of subcutaneous tumors generated by BNL cells in Balb/c milce.

FIG. 2B is a graph depicting growth curves of xenograft tumors generated by BNL cells injected subcutaneously and treated with different reagents in Balb/c mice.

FIG. 3 is an image of representative gross appearance of liver tumors generated by BNL cells following treatment with either PD-1 inhibitor or vaccine alone versus combination, compared to control.

FIG. 4 is a graph depicting the cytotoxicity of murine splenocytes against BNL cells in vitro.

FIG. 5 is a bar graph showing the in vitro cytotoxicity of splenocytes (derived from mice harboring BNL-tumors and treated with vaccine+PD-1 inhibitor) after restimulation against ASPH-expressing 4T1 breast cancer cells.

FIG. 6 are a series of images showing interferon-gamma (IFN-γ) secretion from murine splenocytes after re-stimulation in vitro. The mice of liver cancer models were generated by BNL cells and treated with either vaccine or PD-1 inhibitor alone vs. combination, compared to control.

FIG. 7A are images showing histologic characteristics of liver tumors generated by BNL cells.

FIG. 7B are images showing infiltration of CD3+ T cells into tumors by immunohistochemistry (IHC).

FIG. 7C is a bar graph depicting calculation of the numbers of tumor-infiltrating CD3+ T cells. ***P<0.001.

FIG. 8 is a bar graph depicting that antigen (ASPH) specific antibody (B cell response) stimulated in a murine liver cancer model generated by BNL cells in response to either vaccine or PD-1 inhibitor alone versus combination, compared to control.

FIG. 9 is an image depicting the experimental protocol for an orthotopic murine breast cancer model generated by ASPH expressing 4T1 cells.

FIG. 10 is a graph showing growth curves of primary breast tumors following treatment with either vaccine or PD-1 inhibitor alone vs. combination, compared to control.

FIG. 11 are images showing gross appearance of breast tumors following treatment with either vaccine or PD-1 inhibitor alone vs. combination, compared to control (at day 28).

FIG. 12 is a bar graph depicting reduction in pulmonary metastatic lesions following treatment with either vaccine or PD-1 inhibitor alone versus combination, compared to control.

FIG. 13A is a bar graph showing the reduction in multi-organ metastatic burden following treatment with either vaccine or PD-1 inhibitor alone vs. combination, compared to control.

FIG. 13B is a bar graph depicting the number of mice with versus without metastasis.

FIG. 14 is a graph depicting a dose-dependent antitumor effects of a PD-1 inhibitor on primary tumor growth in vaccinated mice of an orthotopic breast cancer model generated by 4T1 cells.

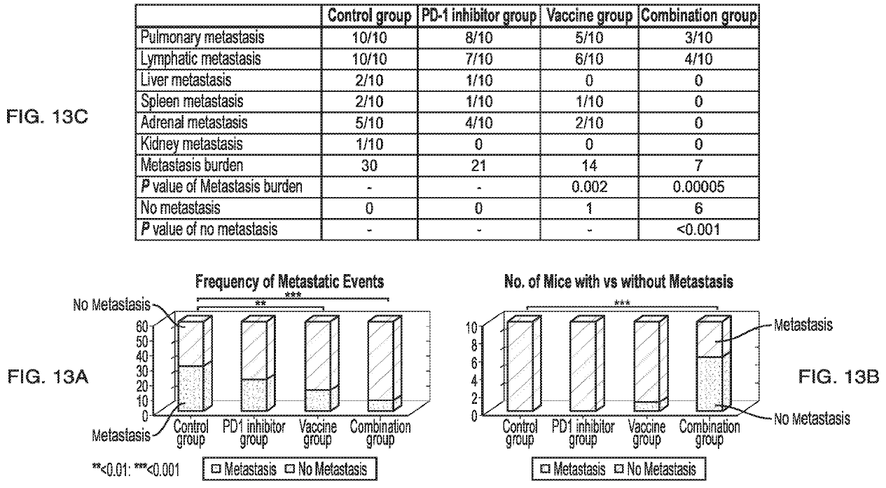

FIG. 13C is a table showing the reduction in multi-organ metastatic burden following treatment with either vaccine or PD-1 inhibitor alone vs. combination, compared to control.

FIG. 15 is a bar graph showing the dose-dependent antitumor effects of a PD-1 inhibitor on pulmonary metastases in vaccinated mice of an orthotopic breast cancer model generated by 4T1 cells.

FIG. 16 is a graph showing the in vitro cytotoxicity of splenocytes against ASPH expressing 4T1 cells.

FIG. 17 are images showing antigen (ASPH) specific T cells activation (as demonstrated by IFNγ secretion) in vaccinated mice of an orthotopic breast cancer model generated by ASPH expressing 4T1 cells.

FIG. 18A are images showing CD3⁺ lymphocytes infiltrated in primary breast tumors following treatment with either vaccine or PD-1 inhibitor alone vs. combination, compared to control; and infiltration of CD3⁺ T cells into tumors by IHC.

FIG. 18B is a bar graph showing the calculation of the number of tumor-infiltrating CD3⁺ T cells.

FIG. 19A are images showing CD3⁺ lymphocytes infiltrated in pulmonary metastases following treatment with either vaccine or PD-1 inhibitor alone vs. combination, compared to control; and infiltration of CD3⁺ T cells into metastatic lesions by IHC.

FIG. 19B is a bar graph showing calculation of the number of tumor-infiltrating CD3⁺ T cells.

FIG. 20A are images showing the characterization of CD8⁺ (effector) CTLs in primary breast cancer tumor and pulmonary metastasis in an orthotopic murine model following treatment with either vaccine or PD-1 inhibitor alone vs. combination, compared to control; and the infiltration of CD3⁺ T cells into primary tumors and pulmonary metastatic lesions by immunohistochemistry IHC.

FIG. 20B is a bar graph depicting calculation of the number of tumor-infiltrating CD3⁺ T cells.

FIG. 20C is a bar graph depicting calculation of the number of tumor-infiltrating CD3⁺ T cells.

FIG. 21A are images showing the characterization of CD45RO⁺ (memory) CTLs in primary breast cancer tumor and pulmonary metastasis in an orthotopic murine model following treatment with either vaccine or PD-1 inhibitor alone vs. combination, compared to control; and infiltration of CD45RO⁺ T cells into primary tumors and pulmonary metastatic lesions by immunohistochemistry IHC.

FIG. 21B is a bar graph depicting the calculation of the number of tumor-infiltrating CD45RO⁺ T cells.

FIG. 21C is a bar graph depicting the calculation of the number of tumor-infiltrating CD45RO⁺ T cells.

FIG. 22 is a bar graph showing antigen (ASPH) specific antibody (B cell response) generated in an orthotopic murine breast cancer model following treatment with either vaccine or PD-1 inhibitor alone vs. combination, compared to control.

### DETAILED DESCRIPTION

Aspartyl asparaginyl β-hydroxylase (ASPH) is a tumor associated antigen (TAA), e.g., a transmembrane protein, present on the cell surface of many types of malignancies and a target for immunotherapy of human cancers. It has been observed that aspartyl ASPH catalyzes the hydroxylation of β carbons in aspartyl and asparaginyl residues found in many signaling molecules (see, for example, Engel, FEBS Lett. 1989; 251:1-7; Jia et al., J. Biol. Chem. 1992; 267:14322-14327; Lavaissiere et al., J. Clin. Invest. 1996; 98:1313-1323; Wang et al., J. Biol Chem. 1991; 266:14004-14010, the entire contents of which are hereby incorporated by reference). Its enzymatic activity depends on the presence of ferric iron and α-ketoglutarate as well as substrates that contain epidermal growth factor (EGF) like repeats (see, for example, Engel, FEBS Lett. 1989; 251:1-7, the entire contents of which are hereby incorporated by reference).

During oncogenesis, ASPH translocates to the cell surface leading to N and C-terminal regions exposed to the extracellular environment and its functions are modulated by the host immune responses. More importantly, the presence of antigenic epitopes that reside on these regions efficiently stimulate T-cell responses specific to tumor cells harboring ASPH (see, for example, Tomimaru et al., Vaccine 2015; 33:1256-1266, the entire contents of which are hereby incorporated by reference). ASPH is a viable target for immunotherapy using a dendritic cell (DC) microparticle vaccine in syngeneic animal models of hepatocellular carcinoma (HCC) and cholangiocarcinoma which has similarities to the λ phage vaccine presented here (see, for example, Noda et al. Hepatology 2012; 55:86-97; Shimoda et al., J. Hepatol. 2012; 56:1129-1135, the entire contents of which are hereby incorporated by reference). The ASPH is highly conserved during mammalian evolution. It is expressed in the embryo during early development, but at birth the gene is silenced only to be reactivated during transformation of normal cells to the malignant phenotype (see, for example, Lavaissiere et al., J. Clin. Invest. 1996; 98:1313-1323; Aihara et al., Hepatology 2014; 60:1302-1313, the entire contents of which are hereby incorporated by reference).

The ASPH directly contributes to oncogenesis since its overexpression stimulates tumor cell proliferation, migration, and invasion (see, for example, Aihara et al., Hepatology 2014; 60:1302-1313; Ince et al., Cancer Res. 2000; 60:1261-1266; Sepe et al., Lab Invest. 2002; 82:881-891, the entire contents of which are hereby incorporated by reference). It was of interest to find the phage vaccination substantially reduced pulmonary metastasis in the orthotopic murine model of breast cancer generated by 4T1 cells. Expression of ASPH in normal tissues is generally extremely low or negligible and/or undetectable except for the placenta, a highly invasive tissue, where gene and protein expression of ASPH approaches the levels found in many malignancies, such as HCC. In this regard, immunohistochemistry (IHC) staining for protein expression and reverse transcription polymerase chain reaction (RT-PCR) for mRNA level have revealed that approximately 85% of hepatitis C virus (HCV) and hepatitis B virus (HBV) related HCC, as well as >95% of cholangiocarcinomas exhibit upregulation of the ASPH gene (see, for example, Noda et al. Hepatology 2012; 55:86-97; Shimoda et al., J. Hepatol. 2012; 56:1129-1135; Aihara et al., Hepatology 2014; 60:1302-1313; Cantarini et al., Hepatology 2006; 44:446-457; Huang et al., PLoS One 2016; 11:e0150336; Iwagami et al., Hepatology 2015, the entire contents of which are hereby incorporated by reference).

The ASPH has been found to exert its biologic effects during oncogenesis partially by the following mechanisms: 1) promotes activation of the Notch signaling cascade; 2) inhibits apoptosis through caspase 3 cleavage; 3) enhances cell proliferation via phosphorylation of RB1; 4) delays cell senescence; and 5) generates cancer stem-like cells (see, for example, Huang et al., PLoS One 2016; 11:e0150336; Iwagami et al., Hepatology 2015; Dong et al., Oncotarget 2015; 6:1231-1248, the entire contents of which are hereby incorporated by reference). The transcriptional regulation of ASPH is controlled by well-known signaling cascades such as insulin (IN)/Insulin receptor substrate 1 (IRS-1)/Rapidly Accelerated Fibrosarcoma (RAF)/Rat Sarcoma (RAS)/Mitogen-Activated Protein (MAP)/extracellular signal-regulated kinases (ERK), IN/IRS-1/Phosphatidylinositol-3-Kinase (PI3 K)/AKT (protein kinase B) and Wingless/Integrated (WNT)/β-catenin signaling (Cantarini et al., Hepatology 2006; 44:446-457; Tomimaru et al., Cancer Lett. 2013; 336:359-369). In this context, ASPH becomes a key molecule that links upstream growth factor signaling pathways to Notch activation and subsequent downstream expression of Notch target genes to participate in oncogenesis, e.g., hepatic oncogenesis. There are also post-translational modifications of ASPH in tumor cells by Glycogen synthase kinase 3β (GSK3β) via phosphorylation of the motifs located in the N-terminal region of the protein (de la Monte et al., Alcohol 2009; 43:225-240).

It is of interest that activation of IN/Insulin-Like Growth Factor 1 (IGF1)/IRS1 mediated pathways, as well as WNT/β-catenin and ASPH/Notch signaling cascades has been shown to be necessary and sufficient for promoting transformation of the normal liver to a malignant phenotype in a double transgenic murine model (see, for example, Chung et al., Cancer Lett. 2016; 370:1-9, the entire contents of which are hereby incorporated by reference). Therefore, inhibition of the expression and function of this putative oncogenic protein could have therapeutic implications.

Immunotherapy is particularly attractive since ASPH: 1) is a transmembrane protein with high expression on cell surface in various maliagnancies; 2) expresses at extremely low/undectable levels in normal human tissues (except placenta); 3) has a defined role in promoting cancer cell proliferation, migration, invasion, and metastasis; 4) high expression confers a poor prognosis of cancer patients, characterized by early disease reoccurrence, reduced overall survival, and a highly undifferentiated aggressive phenotype (see, for example, Maeda et al., Cancer Detect. Prev. 2004; 28:313-318; Wang et al., Hepatology 2010; 52:164-173, the entire contents of which are hereby incorporated by reference).

An immunotherapy approach involves injection of dendritic cells (DCs) loaded with a protein of interest. DCs are specialized antigen-presenting cells (APCs) that recognize/capture, process, and present antigens to T cells to induce and regulate T cell-mediated immunity. DCs are widely used to immunize not only laboratory animals but also tumor-bearing patients. DC vaccine is an antigen primed, activated and loaded, e.g., a purified antigen such as ASPH or antigenic fragments thereof as described herein. The DC vaccine is used to reduce and eliminate, e.g., ASPH-expressing tumors from mammalian subjects, such as human patients. The compositions and methods are also suitable for use in companion animals and livestock, e.g., human, canine, feline, equine, bovine, or porcine subjects. ASPH-expressing tumors include most tumor types such as tumors of gastrointestinal tract (e.g., esophagus, stomach, colon, rectum), pancreas, liver (e.g., cholangiocellular carcinoma, hepatocellular carcinoma), breast, prostate, cervix, ovary, fallopian tube, larynx, (non-small cell) lung, thyroid, gall bladder, kidney, bladder, and brain (e.g., glioblastoma) as well as numerous others. ASPH-expressing tumors include primary tumors that express an increased level of ASPH compared to (adjacent) normal tissues, as well as tumors that arise by metastasis from such ASPH-expressing primary tumors.

Dendritic cells used in the vaccination method are optionally activated ex vivo with a combination of cytokines comprising granulocyte-macrophage colony-stimulating factor (GM-CSF) and IFN-γ prior to administering them to the subject. The latter step yields primed a population of DCs with enhanced capability to stimulate T cell mediated anti-tumor immune responses. An improved method of producing primed DCs is carried out by contacting isolated DCs with an antigen, such as ASPH and antigenic fragments thereof, or a combination of tumor antigens, such as ASPH and alpha-fetoprotein (AFP), and treating DCs to yield a population of matured and activated antigen-presenting cells (APCs). Following the antigen-incubating step, the DCs are matured with the combination of cytokines (cytokine cocktail). For example, the combination comprises GM-CSF and IFN-γ. In other examples, the combination further comprises interleukin-4 (IL-4). Optionally, the combination comprises Cluster of differentiation 40 ligand (CD40L), TNFα, IL1β, IL6, PGE2, agonists for toll like receptor (TLR) ligands (e.g., CL097 (Imidazoquinoline compound R848 derivative), which is a TLR7/8 agonist), or other immune modulators. The DCs are exposed to the combination of cytokines for at least 10 hours (e.g., 12, 24, 36, 40, 48 hours or more). The antigen is in a soluble form or bound to a solid support. For example, the solid support comprises a polystyrene bead such as a biodegradable bead or particle. Dendritic cells are obtained from a subject by known methods such as leukapheresis or cytopheresis.

Dendritic cell vaccines using ASPH are found to cure established hepatocellular carcinoma (HCC) in immunocompetent mice. ASPH-loaded dendritic cell vaccines reduce growth of ASPH-expressing tumors to decrease tumor burden and eradicate tumors in humans as well (see, for example, published US Patent Application 20110076290, the entire contents of which are hereby incorporated by reference).

A prophylactic and therapeutic “phage vaccine” can be used for both cancer prevention and treatment. For example, a cancer vaccine therapy is designed to target a pan-cancer-specific antigen, such as ASPH, using bacteriophage-expressed ASPH fragments. The bacteriophage surface-expressed ASPH is highly immunogenic. Further, bacteriophage delivery of ASPH fragments as vaccine can overcome the problem of self-antigen tolerance by providing antigen presentation and phage adjuvant properties. The bacteriophage may be any one of Lambda, T4, T7, or M13/fl.

Bacteriophage display is a simple way of achieving favorable presentation of peptides to the immune system. Recombinant bacteriophage can prime strong CD8⁺ T lymphocytes (CTLs) responses both in vitro and in vivo against epitopes displayed in multiple copies on their surface, activate T-helper cells and elicit the production of specific antibodies all normally without adjuvant.

Vaccination with lambda phage-displaying cancer specific antigen, such as ASPH, has a number of potential advantages. One of the advantages is display of multiple copies of peptides on the same lambda phage, and once the initial phage display has been made, subsequent production should be far easier and cheaper than the ongoing process of coupling peptides to carriers. There is also good evidence that due to particulate nature, phage-displayed peptides can access both the major histocompatibility complex (MHC) I and MHC II pathway, suggesting lambda phage display vaccines can stimulate both cellular and humoral arms of the immune system, although as extra cellular antigens, it is to be expected that the majority of the responses will be antibody (MHC class II) biased. It has been shown that particulate antigens, and phage in particular, can access the MHC I pathway through cross priming, indicating this process is likely responsible for stimulating a cellular response. This reactivated cellular response mediated by CD8⁺ T cells helps to eliminate the cancer cells. Also, the role of innate immunity in cancer is well established fact. Lambda phage can also act as nonspecific immune stimulators. It is likely that a combination of the foreign DNA (possibly due to the presence of CpG motifs) and the repeating peptide motif of the phage coat are responsible for the nonspecific immune stimulation.

In sum, whole lambda phage particles possess numerous intrinsic characteristics which make them ideal as vaccine delivery vehicles. For use as phage display vaccines, the particulate nature of phage means they should be far easier and cheaper to purify than soluble recombinant proteins. Additionally, the peptide antigen comes already covalently conjugated to an insoluble immunogenic carrier with natural adjuvant properties, without the need for complex chemical conjugation and downstream purification processes which must be repeated with each vaccine batch (see, for example, published US Patent Application 20140271689, the entire contents of which are hereby incorporated by reference).

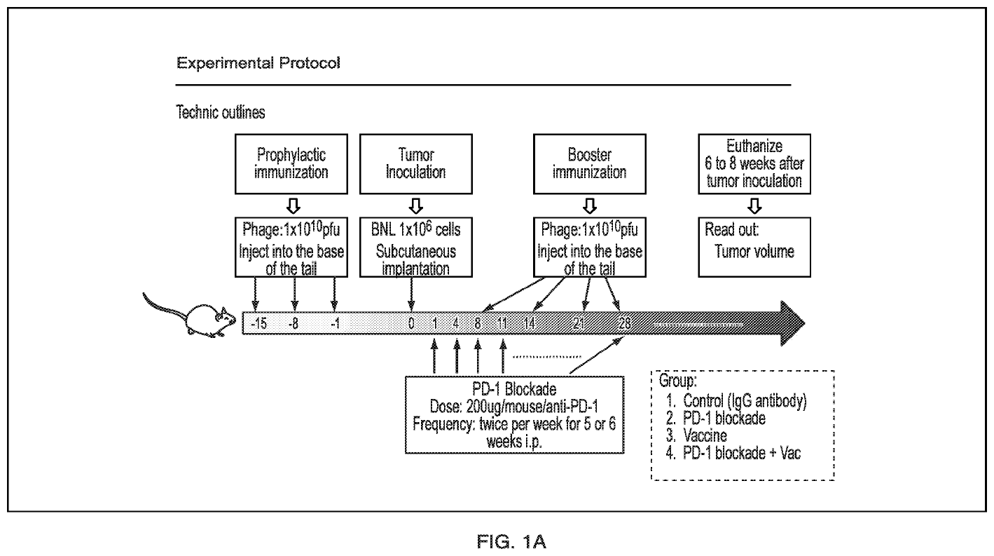

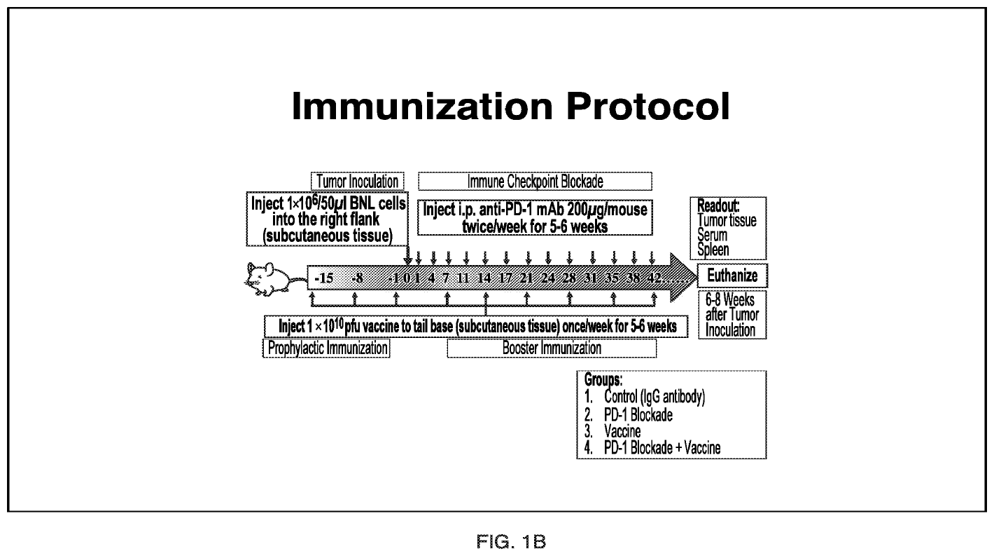

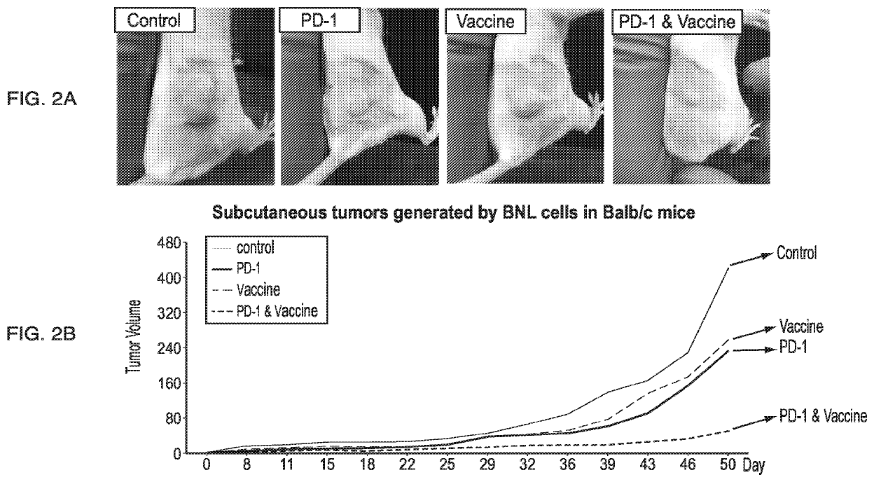

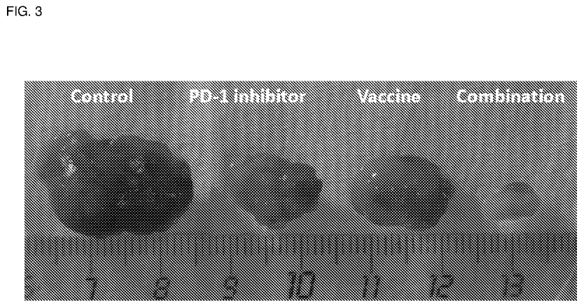

The murine ASPH expressing BNL cell line (ATCC Accession No. TIB-73) produces rapid growth when implanted subcutaneously into syngeneic BALB/c mice. Inoculated animals, which are very severe models of liver cancer (e.g., HCC), may have to be euthanized as early as 4-5 weeks later due to advanced liver tumors as characterized by large size, and poorly differentiated status (see, for example, Shimoda et al., J. Hepatol. 2012; 56:1129-1135, the entire contents of which are hereby incorporated by reference). The level of ASPH expression in BNL induced tumor is robust (see, for example, Shimoda et al., J. Hepatol. 2012; 56:1129-1135, the entire contents of which are hereby incorporated by reference). Using this liver cancer model system (FIG. 1), the question if an immunotherapeutic approach using a dendritic cell (DC) based vaccine containing the entire ASPH peptide would inhibit HCC growth and progression was addressed. Additional studies to investigate whether a lambda 1 phage N terminal ASPH peptide containing vaccine construct would inhibit HCC growth and progression and further the antitumor effect would be amplified by a concurrently or sequentially administered checkpoint inhibitors were performed (FIG. 2A-2B).

The immunization schedule was designed such that the schedule might mimic a hypothetical clinical situation of proposed use by prophylactic vaccination before a surgical resection of HCC tumor followed by booster doses in an attempt to prevent early disease recurrence and to retard the growth and progression of established micro-metastatic disease. There may be a small number of residual tumor cells following surgery that could be effectively abolished or reduced by a λ phage generated immune response (see, for example, Kundig et al., J. Allergy Clin. Immunol. 2006; 117:1470-1476; Sartorius et al., J. Immunol. 2008; 180:3719-3728; Zhikui et al., J. Biomol. Screen 2010; 15:308-313, the entire contents of which are hereby incorporated by reference) and checkpoint inhibitor anti-PD1 antibody.

The method or methods described herein have advantages (enumerated below) over other cancer therapies: (1) stimulates an immune response to a single chemically defined (or purified or isolated) cell surface antigen (ASPH) highly overexpressed in the majority of human solid tumors as shown in Table 1. (2) Generation of this antigen specific B and T cell immune responses can be achieved with vaccines (phage, dendritic cells, DNA based and peptide formulations). (3) This antigen specific immune response can be greatly amplified with the sequential or concurrent administration of immune checkpoint inhibitors (see, for example, Moser et al, J. Immunol. Methods 2010; 353:8-19; Sambrook and Maniatis, Molecular cloning. Second Edition. ed. New York: Cold Spring Harbor Laboratory Press, 1989, the entire contents of which are hereby incorporated by reference). (4) Demonstrates surprising, unanticipated and dramatic inhibition of tumor development, growth and progression as well as metastatic spread to other sites in the body.

This invention has widespread application for the treatment of solid tumors such as hepatocellular (HCC) liver, pancreatic, gastric, esophageal, and triple negative breast cancer, as well as sarcomas, for example, as shown in Table 1 where there may be few, if any, current therapies.

Immune checkpoint blockades are an advanced strategy of cancer management via modulation of immune cell-tumor cell interaction. The checkpoint blockers, such as anti-Programmed cell death protein-1 (PD-1)/Programmed death-ligand 1 (PD-L1) antibodies, are rapidly becoming a highly promising cancer therapeutic approaches that may yield remarkable antitumor responses with relatively limited side effects.

The PD-1/PD-L1 pathway is a good example of the advanced checkpoint molecules that mediates tumor-induced immune suppression. Physiologically, the PD-1/PD-L1 pathway controls the degree of inflammation at locations expressing the antigens to secure normal tissue from damage. When a T cell recognizes the antigen expressed by the MHC complex on the target cell, inflammatory cytokines are produced, initiating the inflammatory process. These cytokines result in PD-L1 expression in the target tissue, binding to the PD-1 protein on the T cell leading to immune tolerance, a phenomenon where the immune system loses the control to mount an inflammatory response, even in the presence of actionable antigens. In certain tumors, most remarkably in melanomas, this protective mechanism is perverted through overexpression of PD-L1; as a result, it circumvents the generation of an immune response to the tumor. PD-1/PD-L1 inhibitors pharmacologically prevent the PD-1/PD-L1 interaction, thus facilitating a positive immune response to kill the tumor cells (see, for example, Alsaab et al., Front Pharmacol. 2017, Aug. 23; 8:561, the entire contents of which are hereby incorporated by reference). PD-1 ligand 2 (PD-L2), the second ligand for PD-1, is also involved in regulating T cell responses. PD-L1 and PD-L2 represent different T-cell antigens, as PD-L1-specific and PD-L2-specific T cells do not cross-react. Activating PD-L2 specific T cells (e.g., by vaccination) provides an attractive strategy for anti-cancer immunotherapy, since PD-L2 specific T cells can directly support anti-cancer immunity by killing of target cells, as well as, indirectly, by releasing pro-inflammatory cytokines into the microenvironment in response to PD-L2-expressing immune suppressive cells (see, for example, Latchman et al., Nat. Immunol. 2001, March; 2(3):261-268; Ahmad et al., Oncoimmunology, 2017, Nov. 1; 7(2):e1390641. eCollection 2018, the entire contents of which are hereby incorporated by reference).

TABLE 1
Percent of human tumors studied that express ASPH by immunohistochemistry.
Percent of human tumors studied that express ASPH by
immuno-histochemistry (IHC)

| Tumor Tissue Type | # Studied | % Positive | Soutte |
|---|---|---|---|
| Hepatocellular Carcinoma | 87 | 92 | PRC + USA |
| Cholangiocarcinoma | 27 | 100 | USA |
| Non-small cell lung cancer | 304 | 82 | PRC + USA |
| Breast cancer | 47 | 85 | PRC + USA |
| Gastric cancer | 51 | 80 | PRC |
| Pancreatic cancer | 109 | 97 | PRC + USA |
| Soft tissue sarcoma | 30 | 84 | PRC |
| Osteosarcoma | 18 | 80 | USA |
| Colon cancer | 41 | 75 | USA |
| Renal cancer | 49 | 83 | PRC |
| Myeloid leukemia | 79 | 88 | PRC |
| Prostate cancer | 46 | 96 | USA |
| Glioblastoma | 15 | 98 | USA |
| Lymphoid leukemia | 80 | 49 | PRC |
| Normal bone marrow | 130 | 0 | PRC |

PRC = People's Republic of China;
USA = United States of America

In recent times, more than four check point inhibitors (e.g., antibodies) have been commercialized for targeting PD-1, PD-L1, and cytotoxic T-lymphocyte associated protein 4 (CTLA-4). The following Table 2 and Table 3 show some selected immunotherapeutic agents, anti-PD-L1 and anti-PD-1, in clinical trials including the possible combination therapy (see, for example, Alsaab et al., Front Pharmacol. 2017, Aug. 23; 8:561, the entire contents of which are hereby incorporated by reference).

TABLE 2
Exemplary immunotherapeutic agents (anti-PD-L1) in clinical trials

| | | | | |
|---|---|---|---|---|
| | | | | Additional |
| CT Number | Phase | Condition | Sponsor | agents |

ATEZOLIZUMAB (PD-L1 INHIBITOR)-APPROVED BY FDA

| | | | | |
|---|---|---|---|---|
| NCT02724878 | II | Non-Clear Cell Kidney | Dana-Farber Cancer | Bevacizumab |
| | | Cancer | Institute | |
| NCT02989584 | I, II | Bladder Cancer, | Memorial Sloan | Gemcitabine |
| | | Metastatic Bladder | Kettering Cancer Center | Cisplatin |
| | | Cancer, Urothelial | | |
| | | Carcinoma | | |
| NCT02302807 | III | Bladder Cancer | Hoffmann-La Roche | Docetaxel |
| | | | | Paclitaxel |
| | | | | Vinflunine |
| NCT02846623 | II | Small Lymphocytic | M.D. Anderson Cancer | Obinutuzumab |
| | | Lymphoma | Center | |
| NCT02788279 | III | Colorectal Cancer | Hoffmann-La Roche | Cobimetinib |
| | | | | Regorafenib |
| NCT02792192 | I, II | High-risk Non-muscle- | Hoffmann-La Roche | Biological: |
| | | invasive Bladder Cancer | | Bacille Calmette- |
| | | (NMIBC) | | Guérin |
| NCT02902029 | II | Malignant Melanoma | University Hospital, | Vemurafenib |
| | | | Essen | Cobimetinib |
| NCT02908672 | III | Melanoma | Hoffmann-La Roche | Vemurafenib |
| NCT03024437 | I, II | Metastatic Cancer | Roberto Pili | Bevacizumab |
| | | | | Entinostat |
| NCT02891824 | III | Ovarian Cancer | ARCAGY/GINECO | Avastin + |
| | | | GROUP | platinum-based |
| | | | | chemotherapy |
| NCT03038100 | III | Ovarian Cancer; | Hoffmann-La Roche | Paclitaxel |
| | | Fallopian Tube Cancer; | | Carboplatin |
| | | Peritoneal Neoplasms | | Bevacizumab |
| NCT02659384 | II | Ovarian Neoplasms | EORTC | Bevacizumab |
| | | | | acetylsalicylic |
| | | | | acid |
| NCT02992912 | II | Patients with Metastatic | Gustave Roussy, Cancer | SABR |
| | | Tumors | Campus, Grand Paris | |
| NCT03016312 | III | Prostatic Neoplasms | Hoffmann-La Roche | Enzalutamide |
| | | Castration-Resistant | | |
| NCT02873195 | II | Recurrent Colorectal | Academic and | Bevacizumab |
| | | Carcinoma; Stage IVA | Community Cancer | Capecitabine |
| | | Colorectal Cancer; Stage | Research, (NCI) | |
| | | IVB Colorectal Cancer | | |
| NCT02926833 | II | Refractory Diffuse | Kite Pharma, Inc. | Biological: KTE- |
| | | Large B Cell Lymphoma | Genentech, Inc. | C19 |
| NCT02748889 | II | Small Cell Lung Cancer | Giuseppe | Etoposide |
| | | (SCLC) | Giaccone,Vanderbilt | MPDL3280A |
| | | | University, Georgetown | |
| | | | University | |
| NCT02763579 | III | Small Cell Lung Cancer | Hoffmann-La Roche | Carboplatin |
| | | | | Etoposide |
| NCT02807636 | III | Urothelial Carcinoma | Hoffmann-La Roche | Carboplatin |
| | | | | Gemcitabine |
| | | | | Cisplatin |
| NCT03029832 | II | Urothelial Carcinoma | Genentech, Inc. | MOXR0916 |
| NCT02875613 | II | Nasopharyngeal Cancer | Assuntina Sacco, M.D., | — |
| | | | Pfizer, University of | |
| | | | California, San Diego | |
| NCT02912572 | II | Metastatic Endometrial | Dana-Farber Cancer | — |
| | | Cancer | Institute, Pfizer | |
| NCT02915523 | I, II | Epithelial Ovarian | Syndax Pharmaceuticals | Entinostat |
| | | Cancer; Peritoneal | Merck KGaA, Pfizer | |
| | | Cancer; Fallopian Tube | | |
| | | Cancer | | |
| NCT02943317 | II | Epithelial Ovarian | Verastem, Inc. | VS-6063 |
| | | Cancer | | |
| NCT02952586 | III | Squamous Cell | Pfizer | Chemo-radiation |
| | | Carcinoma of the Head | | |
| | | and Neck | | |
| NCT02580058 | III | Ovarian Cancer | Pfizer | Biological: PLD |
| NCT02603432 | III | Urothelial Cancer | Pfizer | — |
| NCT02718417 | III | Ovarian Cancer | Pfizer | Carboplatin |
| | | | | paclitaxel |
| NCT02951156 | III | Diffuse Large B-Cell | Pfizer, EMD Serono | Utomilumab |
| | | Lymphoma (DLBCL) | | Rituximab |
| | | | | Azacitidine |
| | | | | Bendamustine |
| | | | | Gemcitabine |
| | | | | Oxaliplatin |

TABLE 3
Exemplary immunotherapeutic agents (anti-PD-1) in clinical trials

| | | | | |
|---|---|---|---|---|
| | | | | Additional |
| CT Number | Phase | Condition | Sponsor | agents |

PIDILIZUMAB (CT001) (ANTI-PD-1)

| | | | | |
|---|---|---|---|---|
| NCT02530125 | II | Stage III Diffuse Large B- | Northwestern | — |
| | | Cell Lymphoma; Stage IV | University; Gateway | |
| | | Diffuse Large B-Cell | for Cancer Research; | |
| | | Lymphoma | National Cancer | |
| | | | Institute (NCI) | |
| NCT02077959 | I/II | Multiple Myeloma | Yvonne Efebera; | lenalidomide |
| | | | CureTech Ltd; Ohio St. | |
| | | | Univ. Comprehensive | |
| | | | Cancer Center | |
| NCT00532259 | III | Lymphoma, Large Cell, | Cure Tech Ltd | — |
| | | Diffuse; Lymphoma, | | |
| | | Mixed Cell, Diffuse; | | |
| | | Primary Mediastinal Large | | |
| | | B-Cell Lymphoma | | |
| NCT01435369 | II | Melanoma; Malignant | Medivation, Inc. | — |
| | | Melanoma | | |
| NCT00532259 | II | Lymphoma, Large Cell, | CureTech Ltd | — |
| | | Diffuse; Lymphoma, | | |
| | | Mixed Cell, Diffuse; | | |
| | | Primary Mediastinal Large | | |
| | | B-Cell Lymphoma | | |
| NCT00890305 | II | Metastatic Colorectal | Medivation, Inc. | FOLFOX |
| | | Cancer | | |
| NCT02077959 | II | Multiple Myeloma | Yvonne Efebera; | Lenalidomide, |
| | | | CureTech Ltd; Ohio | pidilizumab |
| | | | State University | |
| | | | Comprehensive Cancer | |
| | | | Center | |
| NCT02530125 | II | Stage III Diffuse Large B- | Northwestern | Pidilizumab |
| | | Cell Lymphoma; Stage IV | University; Gateway | |
| | | Diffuse Large B-Cell | for Cancer Research; | |
| | | Lymphoma | National Cancer | |
| | | | Institute (NCI) | |
| NCT03002376 | II | Melanoma | Regeneron | — |
| | | | Pharmaceuticals; | |
| | | | Sanofi | |
| NCT02760498 | II | Advanced Cutaneous | Regeneron | — |
| | | Squamous Cell Carcinoma | Pharmaceuticals | |
| NCT02298946 | I | Colorectal Cancer; | National Cancer | Cyclophos- |
| | | Colorectal Neoplasms; | Institute (NCI); | phamide |
| | | Colorectal Carcinoma | National Institutes of | |
| | | | Health Clinical Center | |
| | | | (CC) | |
| NCT01352884 | I | Cancer | MedImmune LLC; | — |
| | | | GlaxoSmithKline | |
| NCT02118337 | I | Select Advanced | MedImmune LLC | MEDI4736 |
| | | Malignancies | | |
| NCT02013804 | I | Advanced Malignancies | MedImmune LLC | — |
| NCT02271945 | I | Relapsed/Refractory | MedImmune LLC | MEDI-551 |
| | | Aggressive B-cell | | |
| | | Lymphomas | | |
| NCT02678260 | I | Advanced Malignancies | Novartis | |
| | | | Pharmaceuticals | |
| NCT02605967 | II | Nasopharyngeal | Novartis | |
| | | Carcinoma | Pharmaceuticals | |
| NCT02608268 | I | Advanced Malignancies | Novartis | MBG453 |
| | | | Pharmaceuticals | |
| NCT02807844 | I | TNBC; Pancreatic | Novartis | MCS110 |
| | | Carcinoma; Melanoma; | Pharmaceuticals | |
| | | Endometrial | Carcinoma | |
| NCT02967692 | III | Melanoma | Novartis | Dabrafenib, |
| | | | Pharmaceuticals | Trametinib |

Despite the huge success and efficacy of the anti-PD-1/PD-L1 therapy response, it is limited to specific types of cancers. For example, immune checkpoint inhibitors thus far have shown little or no activity in the subset of cancers with lower mutation burdens, such as Ewing sarcoma and prostate cancer. In clinical trials of PD-1 inhibitors in unselected populations of patients with colorectal cancer, little to no activity was observed (see, for example, Yarchoan et al., Nat. Rev. Cancer. 2017 April; 17(4):209-222. Epub 2017 Feb. 24; Schumacher and Schreiber, Science 2015 Apr. 3; 348(6230):69-74; Postow et al., N. Engl. J. Med. 2018 Jan. 11; 378(2):158-168, the entire contents of which are hereby incorporated by reference).

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

As used herein, an “isolated” or “purified” small molecule, nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) or polypeptide is free of the genes or sequences that flank it in its naturally-occurring state. Purified also defines a degree of sterility that is safe for administration to a human subject, e.g., lacking infectious or toxic agents. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to achieve a beneficial clinical effect in a mammal. Ultimately, the attending physician or veterinarian decides the appropriate amount and dosage regimen.

The terms “treating” and “treatment” as used herein refer to the administration of an agent or formulation to a clinically symptomatic individual afflicted with an adverse condition, disorder, or disease, so as to effect a reduction in severity and/or frequency of symptoms or signs, eliminate the symptoms or signs and/or their underlying cause, and/or facilitate improvement or remediation of damage. The terms “inhibiting” and “inhibition” of a disease in a subject means preventing or reducing the progression and/or complication of condition, disorder, or disease in the subject. For example, inhibition includes inhibiting adhesion formation.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “subject,” “patient,” “individual,” and the like as used herein are not intended to be limiting and can be generally interchanged. That is, an individual described as a “patient” does not necessarily have a given disease, but may be merely seeking medical advice. The term “subject” as used herein includes any member of the animal kingdom, such as a mammal. In one embodiment, the subject is a human. In another embodiment, the subject is a mouse. For example, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, “treating” encompasses, e.g., inhibition, regression, or stasis of the progression of a disorder. Treating also encompasses the prevention or amelioration of any symptom or symptoms of the disorder. As used herein, “inhibition” of disease progression or a disease complication in a subject means preventing or reducing the disease progression and/or disease complication in the subject.

As used herein, a “symptom” associated with a disorder includes any clinical or laboratory manifestation associated with the disorder, and is not limited to what the subject can feel or observe.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. Such sequences that are at least about 80% identical are said to be “substantially identical.” In some embodiments, two sequences are 100% identical. In certain embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids or nucleotides in length. In certain embodiments, the identity exists over a region that is at least about 50 amino acids in length, or more preferably over a region that is 100 to 500, 100 to 200, 150 to 200, 175 to 200, 175 to 225, 175 to 250, 200 to 225, 200 to 250 or more amino acids in length.

For sequence comparison, typically one sequence acts as a reference sequence, to which test sequences are compared. In various embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In various embodiments, a comparison window is the entire length of one or both of two aligned sequences. In some embodiments, two sequences being compared comprise different lengths, and the comparison window is the entire length of the longer or the shorter of the two sequences. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

In various embodiments, an algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information, as known in the art. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The invention further provides pharmaceutical compositions to be used for treating a tumor in a subject. Exemplary pharmaceutically acceptable carriers include a compound selected from the group consisting of a physiological acceptable salt, poloxamer analogs with carbopol, carbopol/hydroxypropyl methyl cellulose (HPMC), carbopol-methyl cellulose, car-boxymethylcellulose (CMC), hyaluronic acid, cyclodextrin, and petroleum.

The compositions and methods described herein are useful for a subject, wherein the subject is a mammal in need of such treatment. The mammal is, e.g., a human, a primate, a mouse, a rat, a dog, a cat, a horse, as well as livestock or animals grown for food consumption, e.g., cattle, sheep, pigs, chickens, and goats. Preferably, the mammal is a human.

The compositions described herein are administered systemically or topically. In a preferred embodiment, the composition is administrated when medically appropriate.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### EXAMPLES

### Example 1: Sequential and Concurrent Administration of Phage Vaccination Against ASPH and Anti-PD-1 Checkpoint Inhibitor Therapy, when Delivered in Combination, Strikingly and Surprisingly Reduces Tumor Growth and Progression

Tumor growth and progression of tumors, e.g., liver tumors such as HCC, were studied in an art-recognized syngeneic murine model. The experimental protocol is described in FIG. 1. There were four groups of mice (n=10/group): (1) control, (2) PD-1 blockade alone, (3) phage vaccine alone expressing ASPH related peptides, and (4) PD-1 blockade+vaccine. In brief, animals were immunized with phage vaccine expressing N-terminal human ASPH peptides three times spaced one week apart prior to subcutaneous inoculation of BNL murine hepatoma cells followed by PD-1 blockade by anti-PD-1 monoclonal antibody administered twice per week for 5-6 weeks. Tumor size was measured as described (see, for example, Iwagami et al., Heliyon 2017; 3:e00407, the entire contents of which are hereby incorporated by reference). As shown in FIGS. 2A and 2B, there was a striking difference in HCC development and growth when comparing control (untreated) to the PD-1 blockade+vaccine group. FIG. 3 also demonstrates relatively modest anti-tumor effects of vaccine or PD-1 blockade alone on tumor growth, which is intermediate between the control and the combination groups. A striking and synergistic effect was observed from the combination therapy on tumor volume of the excised HCC as from the BALB/c mice. There is very little, if any, growth of the HCC over the observation period in the mice that received the anti-PD-1 antibodies+phage vaccination combination against ASPH.

Antigen Specific Activation of CD8⁺ cytotoxic T lymphocytes (CTL) and CD4⁺ helper T cell are stimulated by phage immunization and PD-1 blockade.

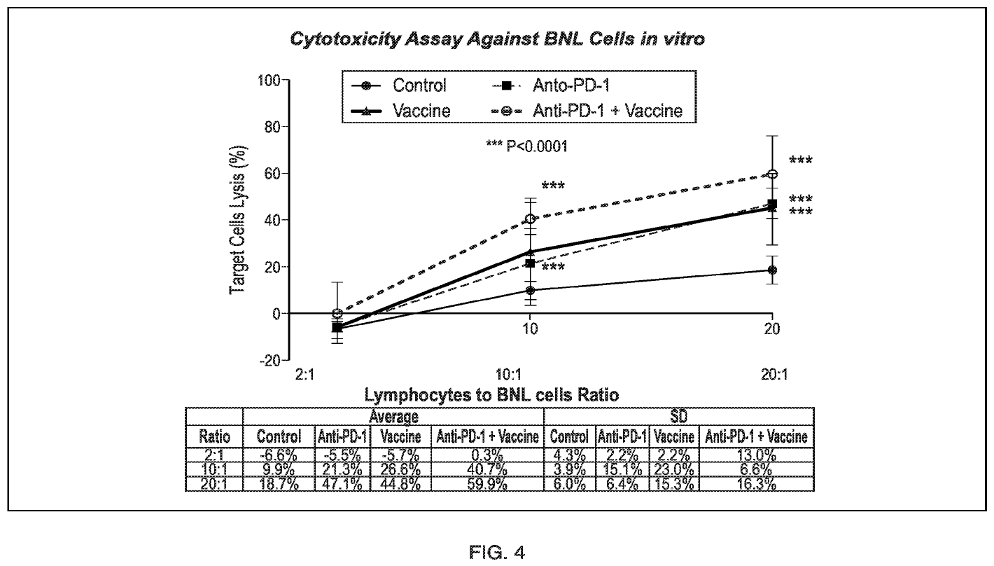

To achieve anti-tumor effects mediated by the endogenous immune system on tumor development and growth, the activation of both CD8⁺ and CD4⁺ cells is required. A cytotoxicity assay that measures CD8⁺ CTL activity was performed as follows: BNL hepatoma cells were seeded into a 96-well plates and allowed to attach for 1 hour followed by the addition of a suspension of splenocytes derived from the various 4 groups described in Example 1, FIG. 1 at a ratio of spenocytes to target cells varying from 2:1 to 20:1 for 4 hours. LDH release from the BNL cells was measured as an indication of cytotoxic activity as described (see, for example, Shimoda et al., J. Hepatol. 2012; 56:1129-1135, the entire contents of which are hereby incorporated by reference). There was a striking increase in CTL activity when comparing the control splenocytes to those obtained from the combination of anti-PD-1+vaccine administration. Anti-PD-1 and vaccine alone generated intermediate responses and the phage vaccination were as effective as anti-PD-1 administration (PD-1 blockade) with respect to BNL hepatoma target cell lysis. (FIG. 4).

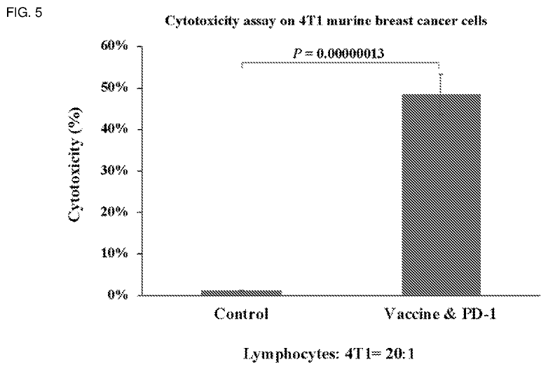

Then, another in vitro cytotoxicity assay was performed using triple negative breast cancer cells, i.e., cancer cells that test negative for estrogen receptors, progesterone receptors, and excess HER2 protein, (e.g., 4T1; ATCC Accession No. CRL-2539) where the splenocytes were derived from the 4 groups of animals described in Example 1, FIG. 1 since 4T1 cells have previously been shown to also express murine ASPH on the cell surface. There was a striking increase of the CD8⁺ CTL activity in the vaccine+PD-1 co-administered group compared to the untreated control. This example showed that splenocytes sensitized to ASPH in vivo can be used to kill other tumor cell types that endogenously express ASPH on the cell surface as demonstrated in FIG. 5.

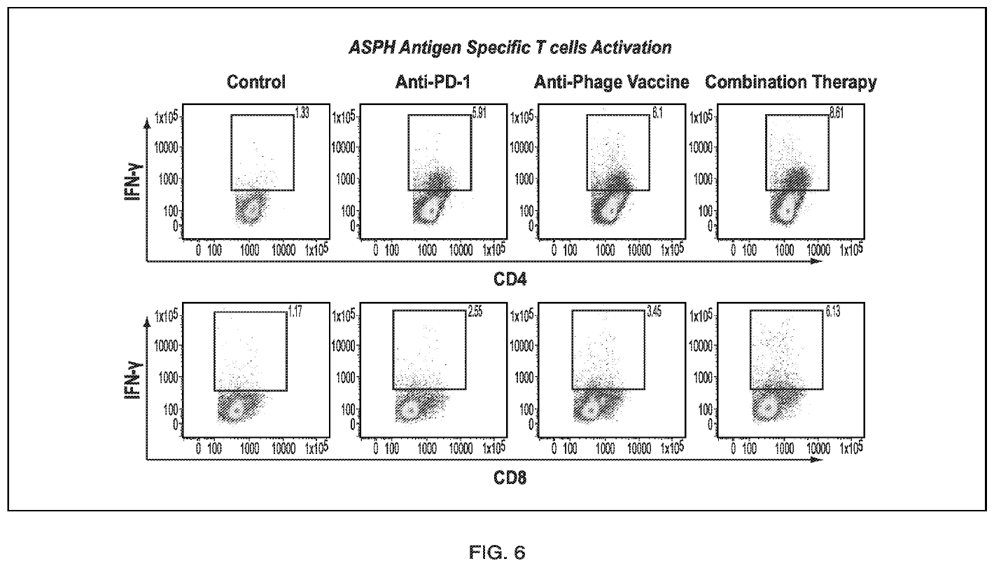

The percent of antigen (ASPH) specific CD4⁺ and CD8⁺ cells that were activated in the splenocyte population by flow cytometry analysis are shown in FIG. 6. There was a substantial increase in ASPH specific CD4⁺ and CD8⁺ activity as measured by the secretion of interferon gamma after stimulation with phage vaccine and recombinant ASPH protein added to the cultured cells. The highest level of activity was observed in the combination therapy compared to either ASPH vaccine or anti-PD-1 administration alone. Therefore, these studies demonstrated that the combination therapy of PD-1 blockade and phage immunization achieved the type of cellular immune responses that are critically required for anti-tumor effects to take place in vivo.

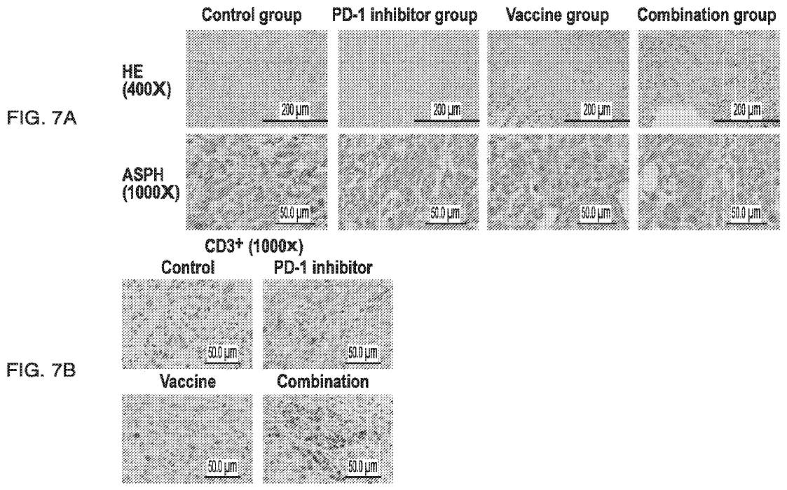

FIG. 7A shows the histologic appearance of the BNL tumors in the 4 groups. The ASPH expression in as measured by immunohistochemistry (IHC) is robust and equal in all tumor treated groups. FIG. 7B shows infiltration of CD3⁺ T cells (brown color) into the tumors from animals treated with either anti-PD1 inhibitor or vaccine alone, as well as combination of both of the PD-1 inhibitor and vaccine administration, as compared to control. FIG. 7C shows more significant synergistic effects of the combination than either vaccine or PD-1 inhibitor alone, as compared to control. There is strikingly and surprisingly enhanced infiltration of CD3⁺ T cells (TILs) in the combination treated tumor. This finding explains, in part, the dramatic decrease in tumor growth and progression with the combination therapy.

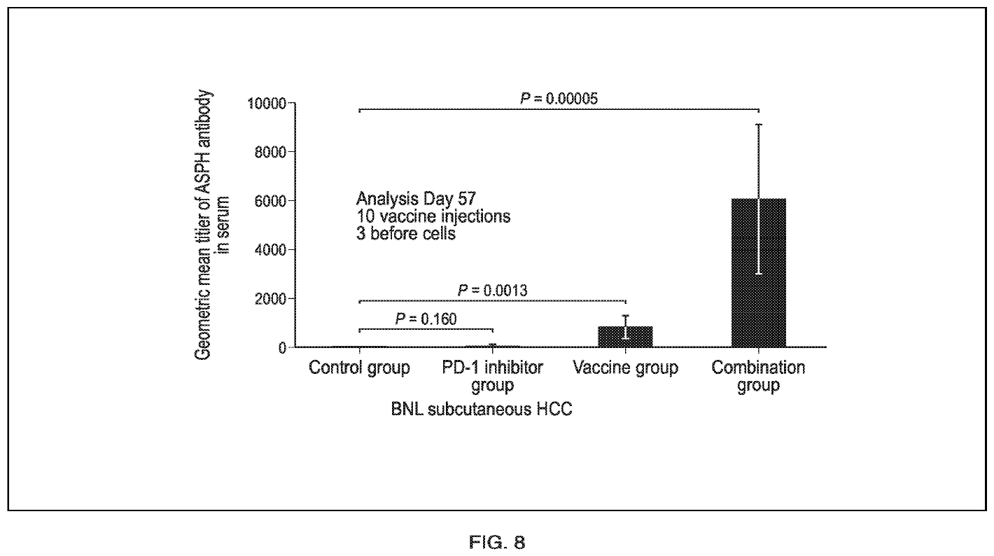

Importantly, antigen (ASPH) specific antibody (B cell response) has been detected in the mice from vaccine and combination groups of liver cancer models generated by ASPH expressing BNL cells (FIG. 8).

### Example 2: Sequential and Concurrent Administration of Phage Vaccination Against ASPH and Anti-PD-1 Checkpoint Inhibitor Therapy, when Delivered in Combination, Strikingly and Surprisingly Reduces Breast Tumor Growth and Progression in a Syngeneic Murine Model

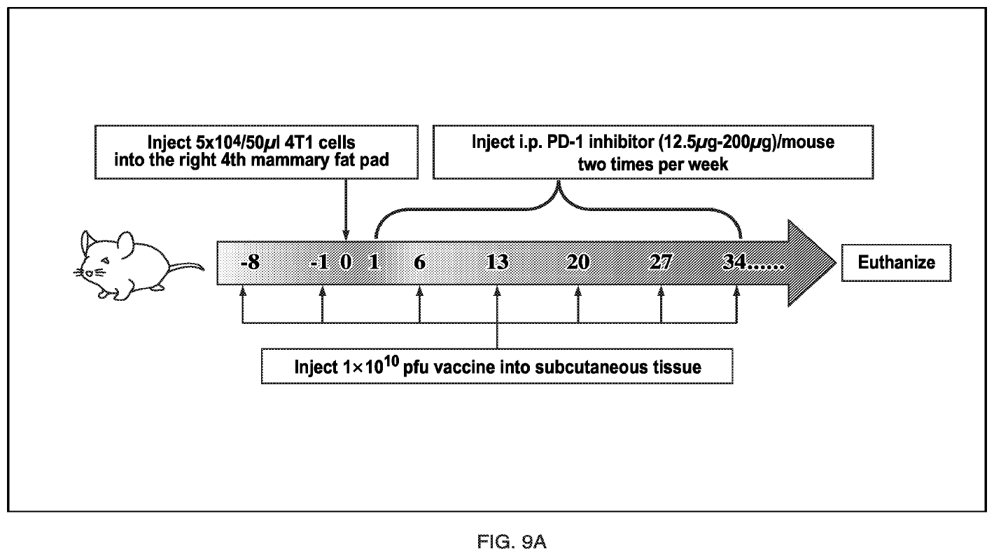

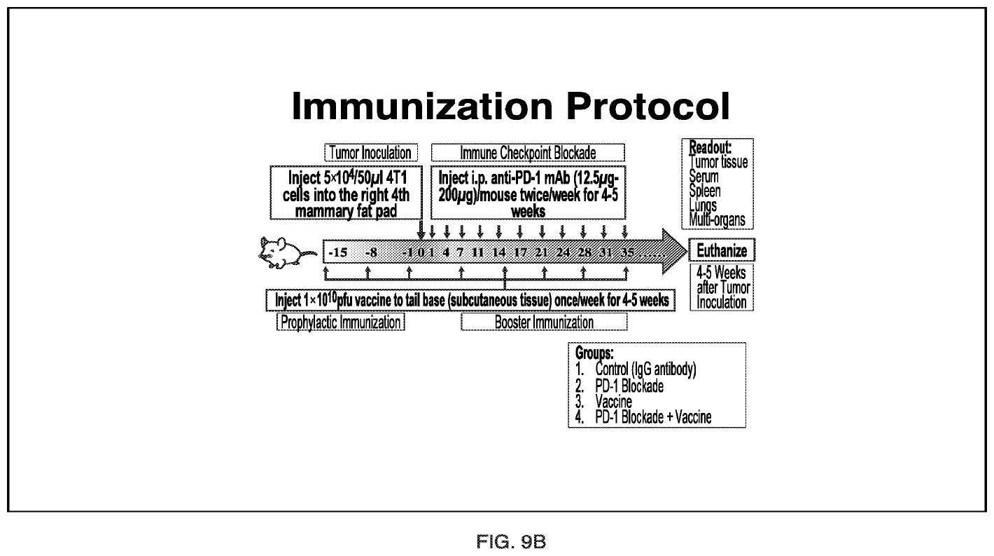

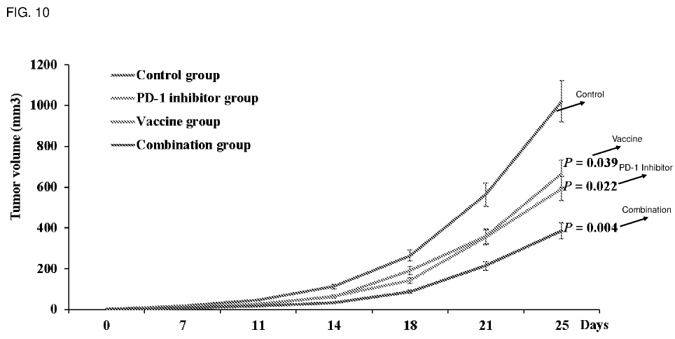

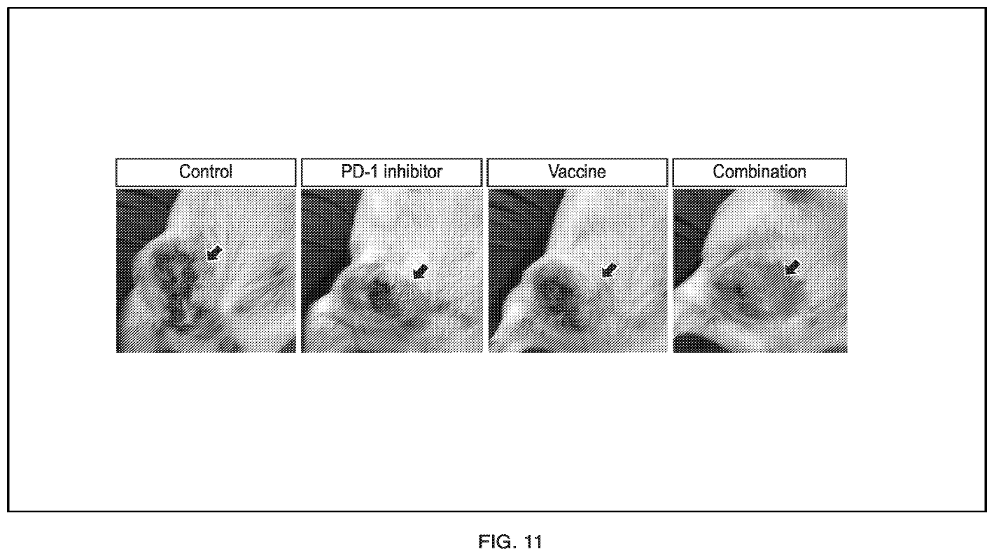

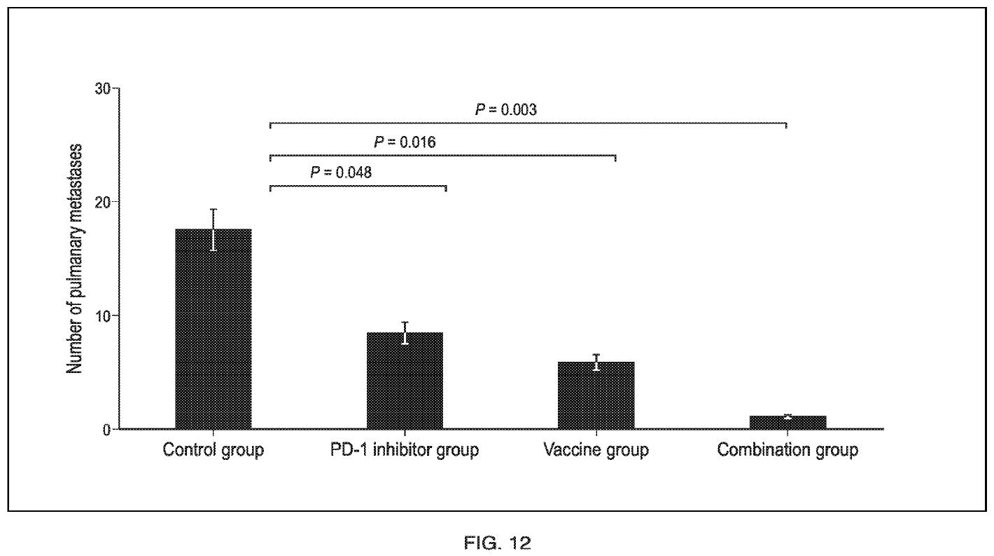

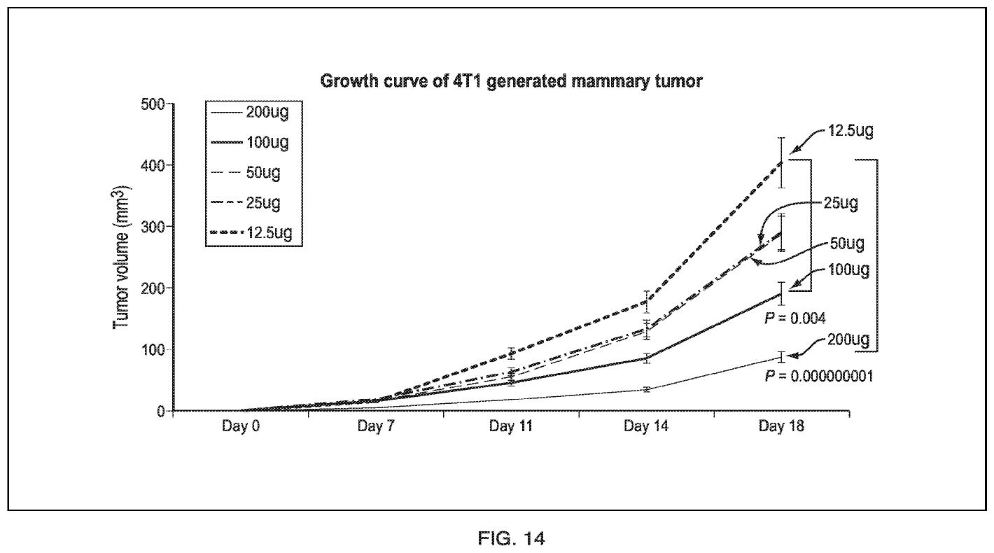

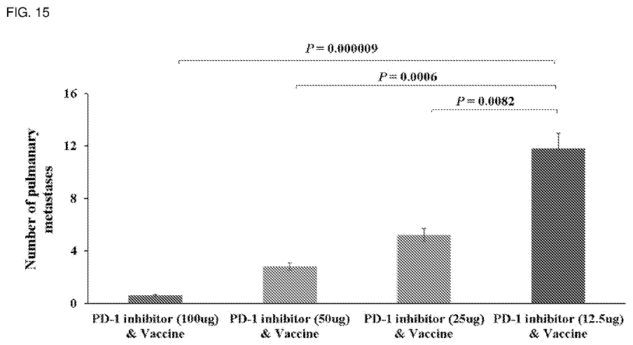

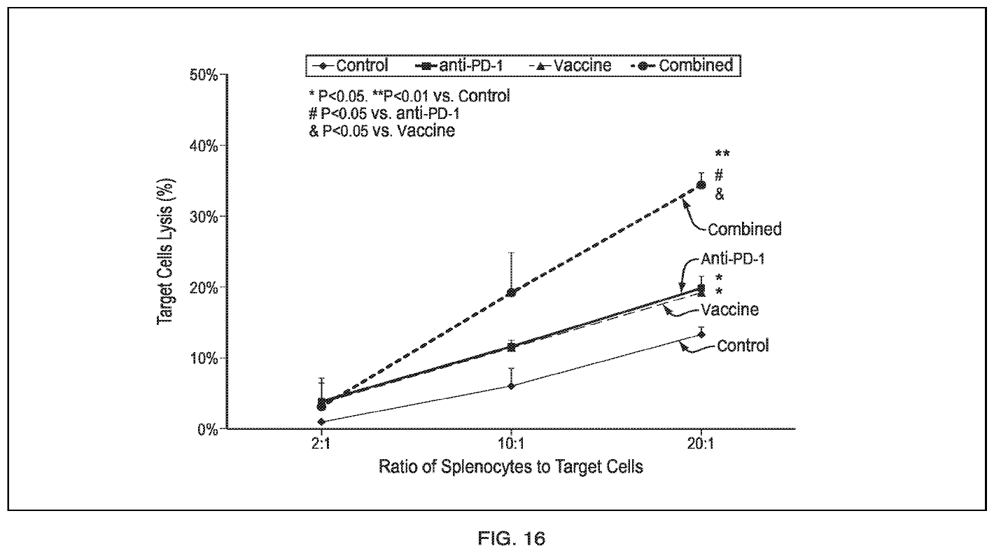

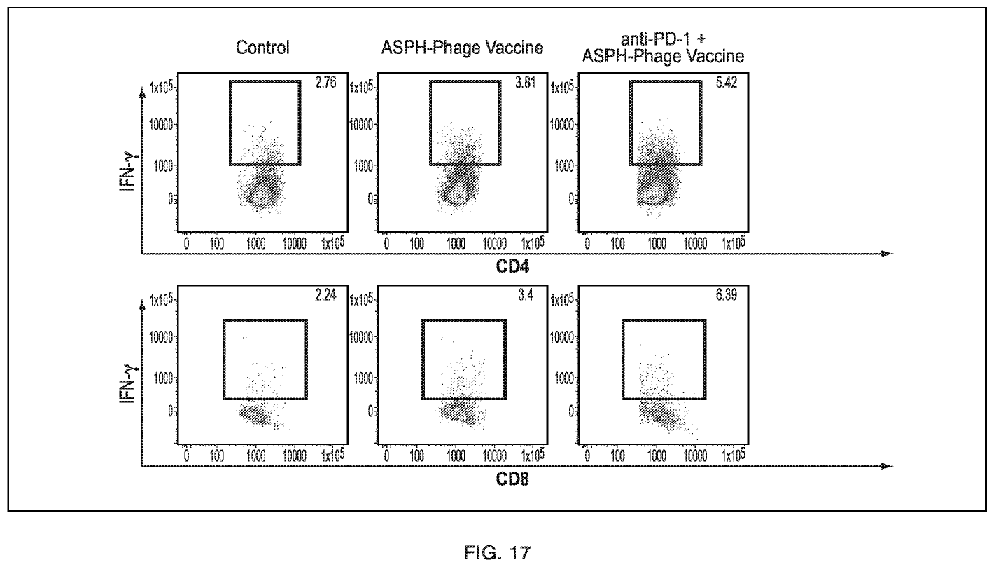

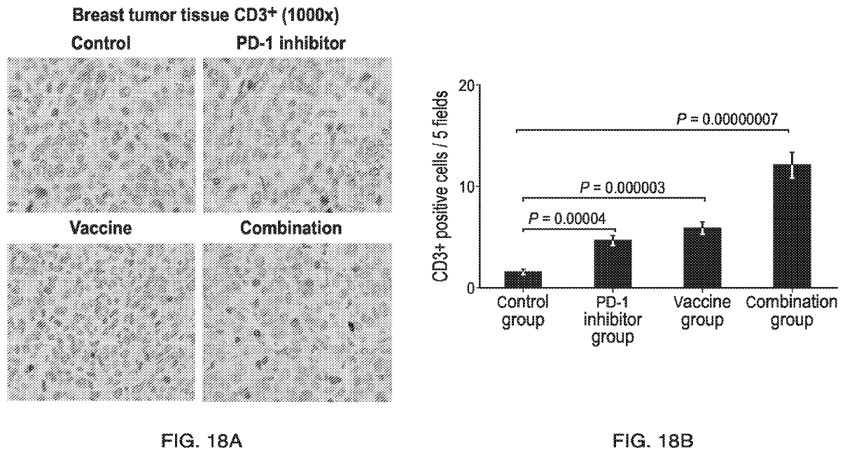

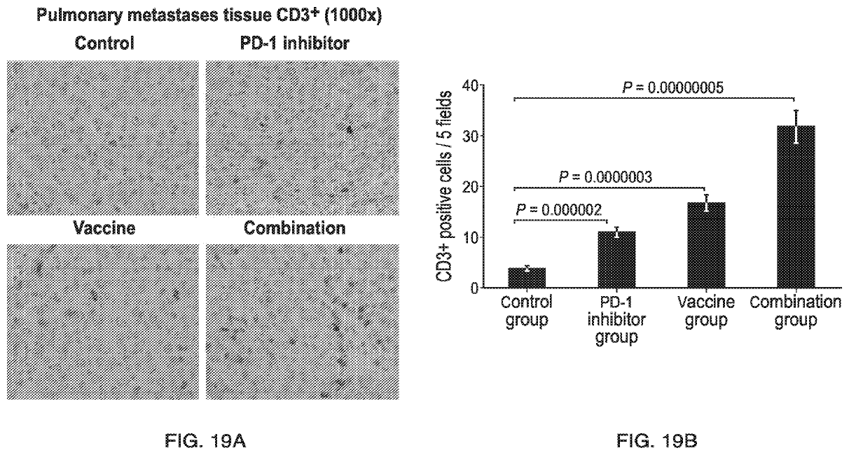

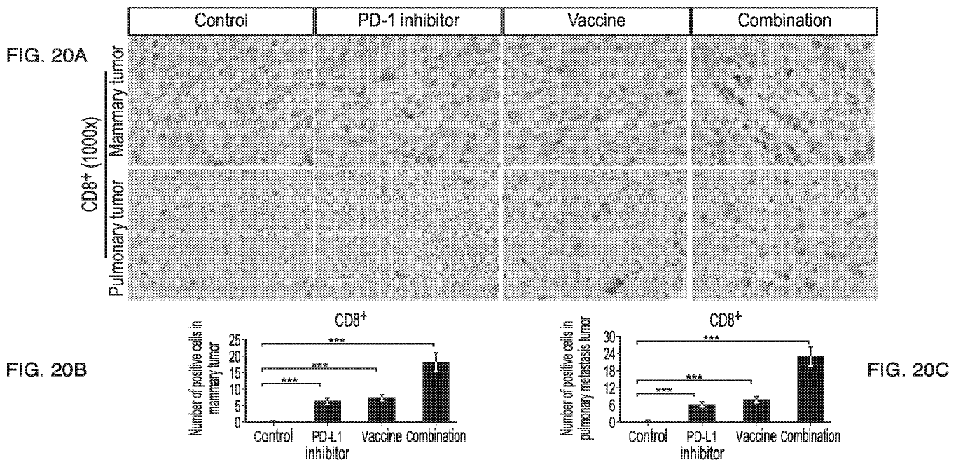

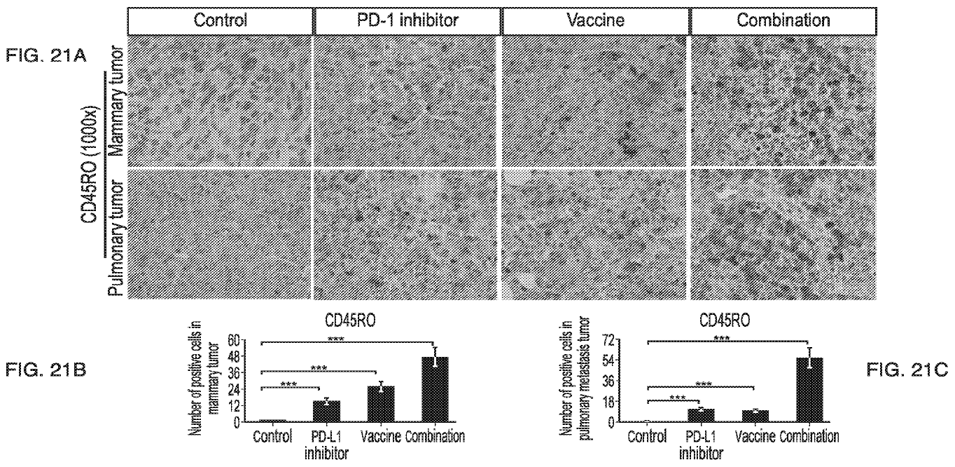

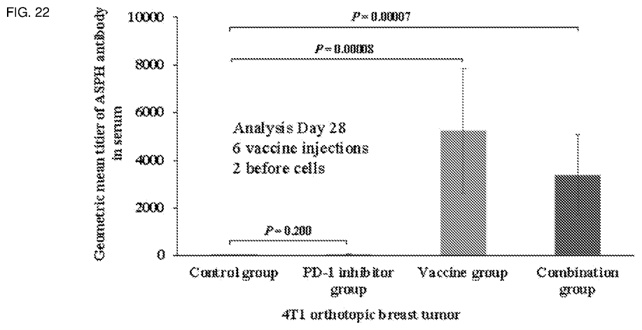

An art-recognized syngeneic murine model was used in the experiments described below. The experimental protocol is shown in FIG. 9. There were four groups of mice (n=10/group) as the following: 1) control, 2) PD-1 blockade (murine anti-PD-1 mAb) alone, 3) lambda 1 phage vaccine expressing N terminal ASPH peptides (SEQ ID NO: 47 in Table 4), and 4) PD-1 blockade+vaccine. Animals were immunized with phage vaccine expressing N-terminal human ASPH peptides three times spaced one week apart prior to orthotopic (mammary fat pad) inoculation of 4T1 murine breast cancer cells followed by PD-1 blockade by anti-PD-1 monoclonal antibody administered twice per week for 5-6 weeks. Tumor size was measured as described (see, for example, Iwagami et al., Heliyon 2017; 3:e00407, the entire contents of which are hereby incorporated by reference). As shown in the graph of depicted in FIG. 10, there was a striking difference in breast cancer development and growth when comparing control (untreated) to the PD-1 blockade+vaccine group. FIG. 11 also demonstrates relatively modest anti-tumor effects of vaccine or PD-1 blockade alone on tumor growth, which is intermediate between the control and the combination groups. Note the striking and unexpected effects of the combination therapy on both primary tumor growth and pulmonary metastasis of breast cancer from the BALB/c mouse (FIG. 12). There are dramatically reduced growth, progression and multiple-organ metastases (at different and distant sites, such as liver, lymph nodes, spleen, adrenal gland, and kidney) of breast cancer over the observation period in the mice that received the anti-PD-1 antibodies+phage vaccination against ASPH (FIGS. 13A-13C). Furthermore, dose-dependent anti-tumor effects of the PD-1 inhibitor have been observed in vaccinated mice (FIGS. 14 and 15). The high dose (200 μg) PD-1 inhibitor has demonstrated paramount inhibitory effects on both primary tumor growth and pulmonary metastasis.

Antigen Specific Activation of CD8⁺ Cytotoxic T lymphocytes (CTL) and CD4⁺ helper T cell are stimulated by lambda 1 phage immunization and PD-1 blockade as demonstrated by flow cytometry, in vitro cytotoxicity, immunohistochemistry and ELISA.

To achieve anti-tumor effects mediated by the endogenous immune system on tumor development and growth, the activation of both CD8⁺ and CD4⁺ cells is required. A cytotoxicity assay that measures CD8⁺ CTL activity was performed as follows: 4T1 cells were seeded into a 96-well plates and allowed to attach for 1 hour followed by the addition of a suspension of splenocytes derived from the various 4 groups described in Example 2, FIG. 9 at a ratio of spenocytes to target cells varying from 2:1 to 20:1 for 4 hours. LDH release from 4T1 cells was measured as an indication of cytotoxic activity as described (see, for example, Shimoda et al., J. Hepatol. 2012; 56:1129-1135, the entire contents of which are hereby incorporated by reference). There was a strikingly synergistic increase in CTL activity when comparing the control splenocytes to those obtained from the combination of anti-PD-1+vaccine administration. For example, the combined effect of the vaccine construct for an immunization against a purified tumor antigen and checkpoint inhibitor is greater than the sum of the effects of the vaccine construct for an immunization against a purified tumor antigen and the checkpoint inhibitor when each agent is used separately. Anti-PD-1 and vaccine alone generated intermediate responses and the phage vaccination were as effective as anti-PD-1 administration (PD-1 blockade) with respect to 4T1 target cell lysis. (FIG. 16).

The percentages of antigen (ASPH) specific CD4⁺ and CD8⁺ cells that were activated in the splenocyte population by flow cytometry analysis are shown in FIG. 17. There was a substantial increase in ASPH specific CD4⁺ and CD8⁺ activity as measured by the secretion of IFNγ after stimulation with phage vaccine and recombinant ASPH protein added to the cultured cells. The highest level of activity was observed in the combination therapy compared to either ASPH vaccine or anti-PD-1 administration alone. Therefore, these studies demonstrate that the combination therapy of PD-1 blockade and phage immunization achieved the type of cellular immune responses that are critically required for anti-tumor effects to take place in vivo.

FIGS. 18A and 18B show infiltration of CD3⁺ T cells (brown color) into the primary tumors from control, anti-PD-1 inhibitor alone, vaccine group alone and combination of PD-1 inhibitor and vaccine administration. FIG. 19 shows substantial synergistic effects of the combination on pulmonary metastasis, more profound than either vaccine or PD-1 inhibitor alone, compared to control. There is strikingly, synergistically enhanced infiltration of CD8⁺ effector cytotoxic CTLs (FIGS. 20A and 20B) and CD45RO⁺ memory CTLs (FIGS. 21A and 21B) among the CD3+ T cells (TILs) into both primary tumors and pulmonary metastases in the combination group. This finding explains, in part, the dramatic, synergistic decrease in tumor growth and progression with the combination therapy.

Importantly, antigen (ASPH) specific antibody (B cell response) has been detected in the mice from vaccine and combination groups of breast cancer models generated by 4T1 cells (FIG. 22).

TABLE 4
Sequences

| Name | SEQ ID NO: | SEQUENCE |
|---|---|---|
| p52 | SEQ ID NO: 1 | TSFFTWFMVIALLGVWTSVA |
| p103 | SEQ ID NO: 2 | AKVLLGLKERSTSEP |
| p148 | SEQ ID NO: 3 | KEQIQSLLHEMVHAEHVEG |
| p322 | SEQ ID NO: 4 | QKAKVKKKKPKLLNKF |
| p415 | SEQ ID NO: 5 | PADLLKLSLKRRSDRQQF |
| p427 | SEQ ID NO: 6 | SDRQQFLGHMRGSLLTLQ |
| p437 | SEQ ID NO: 7 | RGSLLTLQRLVQLFPN |
| p443 | SEQ ID NO: 8 | LQRLVQLFPNDTSLKN |
| p492 | SEQ ID NO: 9 | VHYGFILKAQNKIAESIP |
| p557 | SEQ ID NO: 10 | ASVWQRSLYNVNGLKAQPWW |
| p581 | SEQ ID NO: 11 | TGYTELVKSLERNWKLI |
| p588 | SEQ ID NO: 12 | KSLERNWKLIRDEGLAVMDK |
| p725 | SEQ ID NO: 13 | HEVWQDASSFRLIF |
| p731 | SEQ ID NO: 14 | ASSFRLIFIVDVWHPEL |
| VDVWHPELTP | SEQ ID NO: 15 | VDVWHPELTPQQRRSLPAI |
| QQRRSLPAI | | |
| ASPH48 | SEQ ID NO: 16 | GLSGTSFFT |
| ASPH53 | SEQ ID NO: 17 | SFFTWFMVI |
| ASPH58 | SEQ ID NO: 18 | FMVIALLGV |
| ASPH62 | SEQ ID NO: 19 | ALLGVWTSV |
| ASPH72 | SEQ ID NO: 20 | VVWFDLVDY |
| ASPH79 | SEQ ID NO: 21 | DYEEVLGKL |
| ASPH81 | SEQ ID NO: 22 | EEVLGKLGI |
| ASPH252 | SEQ ID NO: 23 | TDDVTYQVY |
| ASPH258 | SEQ ID NO: 24 | QVYEEQAVY |
| ASPH261 | SEQ ID NO: 25 | EEQAVYEPL |
| ASPH371 | SEQ ID NO: 26 | YPQSPRARY |
| ASPH374 | SEQ ID NO: 27 | SPRARYGKA |
| ASPH406 | SEQ ID NO: 28 | QEVASLPDV |
| ASPH411 | SEQ ID NO: 29 | LPDVPADLL |
| ASPH475 | SEQ ID NO: 30 | KVYEEVLSV |
| ASPH478 | SEQ ID NO: 31 | EEVLSVTPN |
| ASPH484 | SEQ ID NO: 32 | TPNDGFAKV |
| ASPH488 | SEQ ID NO: 33 | GFAKVHYGF |
| ASPH491 | SEQ ID NO: 34 | KVHYGFILK |
| ASPH503 | SEQ ID NO: 35 | KIAESIPYL |
| ASPH521 | SEQ ID NO: 36 | GTDDGRFYF |
| ASPH537 | SEQ ID NO: 37 | RVGNKEAYK |
| ASPH557 | SEQ ID NO: 38 | ASVWQRSLY |
| ASPH563 | SEQ ID NO: 39 | SLYNVNGLK |
| ASPH582 | SEQ ID NO: 40 | GYTELVKSL |
| ASPH611 | SEQ ID NO: 41 | LFLPEDENL |
| ASPH681 | SEQ ID NO: 42 | GPTNCRLRM |
| ASPH693 | SEQ ID NO: 43 | LVIPKEGCK |
| ASPH701 | SEQ ID NO: 44 | KIRCANETR |
| ASPH711 | SEQ ID NO: 45 | WEEGKVLIF |
| Human ASPH | SEQ ID NO: 46 | MAQRKNAKSSGNSSSSGSGSGSTSAGSSSPGAR |
| amino acid | | RETKHGGHKNGRKGGLSGTSFFTWFMVIALLG |
| sequence | | VWTSVAVVWFDLVDYEEVLGKLGIYDADGDG |
| | | DFDVDDAKVLLGLKERSTSEPAVPPEEAEPHTE |
| | | PEEQVPVEAEPQNIEDEAKEQIQSLLHEMVHAE |
| | | HVEGEDLQQEDGPTGEPQQEDDEFLMATDVD |
| | | DRFETLEPEVSHEETEHSYHVEETVSQDCNQD |
| | | MEEMMSEQENPDSSEPVVEDERLHHDTDDVT |
| | | YQVYEEQAVYEPLENEGIEITEVTAPPEDNPVE |
| | | DSQVIVEEVSIFPVEEQQEVPPETNRKTDDPEQK |
| | | AKVKKKKPKLLNKFDKTIKAELDAAEKLRKRG |
| | | KIEEAVNAFKELVRKYPQSPRARYGKAQCEDD |
| | | LAEKRRSNEVLRGAIETYQEVASLPDVPADLLK |
| | | LSLKRRSDRQQFLGHMRGSLLTLQRLVQLFPN |
| | | DTSLKNDLGVGYLLIGDNDNAKKVYEEVLSVT |
| | | PNDGFAKVHYGFILKAQNKIAESIPYLKEGIESG |
| | | DPGTDDGRFYFHLGDAMQRVGNKEAYKWYE |
| | | LGHKRGHFASVWQRSLYNVNGLKAQPWWTP |
| | | KETGYTELVKSLERNWKLIRDEGLAVMDKAK |
| | | GLFLPEDENLREKGDWSQFTLWQQGRRNENA |
| | | CKGAPKTCTLLEKFPETTGCRRGQIKYSIMHPG |
| | | THVWPHTGPTNCRLRMHLGLVIPKEGCKIRCA |
| | | NETRTWEEGKVLIFDDSFEHEVWQDASSPRLIFI |
| | | VDVWHPELTPQQRRSLPAI |
| the first third | SEQ ID NO: 47 | MAQRKNAKSSGNSSSSGSGSGSTSAGSSSPGAR |
| of the human | | RETKHGGHKNGRKGGLSGTSFFTWFMVIALLG |
| ASPH amino | | VWTSVAVVWFDLVDYEEVLGKLGIYDADGDG |
| acid sequence | | DFDVDDAKVLLGLKERSTSEPAVPPEEAEPHTE |
| | | PEEQVPVEAEPQNIEDEAKEQIQSLLHEMVHAE |
| | | HVEGEDLQQEDGPTGEPQQEDDEFLMATDVD |
| | | DRFETLEPEVSHEETEHSYHVEETVSQDCNQD |
| | | MEEMMSEQENPDSSEPVVEDERLHHDTD |
| the last third of | SEQ ID NO: 48 | ESIPYLKEGIESGDPGTDDGRFYFHLGDAMQRV |
| the human | | GNKEAYKWYELGHKRGHFASVWQRSLYNVN |
| ASPH amino | | GLKAQPWWTPKETGYTELVKSLERNWKLIRDE |
| acid sequence | | GLAVMDKAKGLFLPEDENLREKGDWSQFTLW |
| | | QQGRRNENACKGAPKTCTLLEKFPETTGCRRG |
| | | QIKYSIMHPGTHVWPHTGPTNCRLRMHLGLVIP |
| | | KEGCKIRCANETRTWEEGKVLIFDDSFEHEVW |
| | | QDASSFRLIFIVDVWHPELTPQQRRSLPAI |
| Human ASPH | SEQ ID NO: 49 | cggaccgtgcaatggcccagcgtaagaatgccaagagcagcggcaaca |
| nucleotide | | gcagcagcagcggctccggcagcggtagcacgagtgcgggcagcagc |
| sequence | | agccccggggcccggagagagacaaagcatggaggacacaagaatgg |
| | | gaggaaaggcggactctcgggaacttcattcttcacgtggtttatggtgatt |
| | | gcattgctgggcgtctggacatctgtagctgtcgtttggtttgatcttgttgac |
| | | tatgaggaagttctaggaaaactaggaatctatgatgctgatggtgatgga |
| | | gattttgatgtggatgatgccaaagttttattaggacttaaagagagatctact |
| | | tcagagccagcagtcccgccagaagaggctgagccacacactgagccc |
| | | gaggagcaggttcctgtggaggcagaaccccagaatatcgaagatgaag |
| | | caaaagaacaaattcagtcccttctccatgaaatggtacacgcagaacatg |
| | | ttgagggagaagacttgcaacaagaagatggacccacaggagaaccac |
| | | aacaagaggatgatgagtttcttatggcgactgatgtagatgatagatttga |
| | | gaccctggaacctgaagtatctcatgaagaaaccgagcatagttaccacg |
| | | tggaagagacagtttcacaagactgtaatcaggatatggaagagatgatgt |
| | | ctgagcaggaaaatccagattccagtgaaccagtagtagaagatgaaag |
| | | attgcaccatgatacagatgatgtaacataccaagtctatgaggaacaagc |
| | | agtatatgaacctctagaaaatgaagggatagaaatcacagaagtaactg |
| | | ctccccctgaggataatcctgtagaagattcacaggtaattgtagaagaag |
| | | taagcatttttcctgtggaagaacagcaggaagtaccaccagaaacaaata |
| | | gaaaaacagatgatccagaacaaaaagcaaaagttaagaaaaagaagc |
| | | ctaaacttttaaataaatttgataagactattaaagctgaacttgatgctgcag |
| | | aaaaactccgtaaaaggggaaaaattgaggaagcagtgaatgcatttaaa |
| | | gaactagtacgcaaataccctcagagtccacgagcaagatatgggaagg |
| | | cgcagtgtgaggatgatttggctgagaagaggagaagtaatgaggtgcta |
| | | cgtggagccatcgagacctaccaagaggtggccagcctacctgatgtcc |
| | | ctgcagacctgctgaagctgagtttgaagcgtcgctcagacaggcaacaa |
| | | tttctaggtcatatgagaggttccctgcttaccctgcagagattagttcaact |
| | | atttcccaatgatacttccttaaaaaatgaccttggcgtgggatacctcttgat |
| | | aggagataatgacaatgcaaagaaagtttatgaagaggtgctgagtgtga |
| | | cacctaatgatggctttgctaaagtccattatggcttcatcctgaaggcaca |
| | | gaacaaaattgctgagagcatcccatatttaaaggaaggaatagaatccg |
| | | gagatcctggcactgatgatgggagattttatttccacctgggggatgccat |
| | | gcagagggttgggaacaaagaggcatataagtggtatgagcttgggcac |
| | | aagagaggacactttgcatctgtctggcaacgctcactctacaatgtgaat |
| | | ggactgaaagcacagccttggtggaccccaaaagaaacgggctacaca |
| | | gagttagtaaagtctttagaaagaaactggaagttaatccgagatgaaggc |
| | | cttgcagtgatggataaagccaaaggtctcttcctgcctgaggatgaaaac |
| | | ctgagggaaaaaggggactggagccagttcacgctgtggcagcaagga |
| | | agaagaaatgaaaatgcctgcaaaggagctcctaaaacctgtaccttacta |
| | | gaaaagttccccgagacaacaggatgcagaagaggacagatcaaatatt |
| | | ccatcatgcaccccgggactcacgtgtggccgcacacagggcccacaa |
| | | actgcaggctccgaatgcacctgggcttggtgattcccaaggaaggctgc |
| | | aagattcgatgtgccaacgagaccaggacctgggaggaaggcaaggtg |
| | | ctcatctttgatgactcctttgagcacgaggtatggcaggatgcctcatcttt |
| | | ccggctgatattcatcgtggatgtgtggcatccggaactgacaccacagc |
| | | agagacgcagccttccagcaatttagcatgaattcatgcaagcttgggaaa |
| | | ctctggagaga |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

1. A composition for immunotherapy for treating a tumor in a subject comprising: sequential and/or concurrent administration of a vaccine construct for immunization against a tumor antigen, said composition comprising a purified tumor antigen and an immune checkpoint inhibitor, said tumor being characterized as comprising a low frequency of neoantigen expression, and a checkpoint inhibitor for treating a tumor in said subject, wherein the composition potentiates an anti-tumor immune response without inducing autoimmunity in said subject.

2. The composition of claim 1, wherein said antigen is an aspartate beta-hydroxylase (ASPH) or an antigen fragment thereof.

3. The composition of claim 2, wherein said vaccine construct expresses a purified ASPH antigen.

4. The composition of claim 3, wherein said purified ASPH antigen comprises a purified N-terminal ASPH peptide (SEQ ID NO: 47).

5. The composition of claim 3, wherein said purified ASPH antigen comprises a purified C-terminal ASPH peptide (SEQ ID NO: 48).

6. The composition of claim 3, wherein said purified ASPH antigen is a purified peptide selected from the group consisting of SEQ ID NOs: 1-45.

7. The composition of claim 3, wherein said purified ASPH antigen comprises a human leukocyte antigen (HLA) class II restricted sequence of TGYTELVKSLERNWKLI (SEQ ID NO: 11), or an HLA class I restricted sequence of YPQSPRARY (SEQ ID NO:26).

8. (canceled)

9. The composition of claim 1, wherein said vaccine construct comprises a phage vaccine or a dendritic cell vaccine.

10. The composition of claim 9, wherein said phage vaccine is a lambda phage-based vaccine and wherein said dendritic cell vaccine comprises an isolated ASPH-loaded dendritic cell.

11. The composition of claim 1, wherein said checkpoint inhibitor is a Programmed cell death protein-1 (PD-1) inhibitor.

12. The composition of claim 11, wherein said PD-1 inhibitor is a PD-1 inhibitory antibody, a PD-1 inhibitory nucleic acid, a PD-1 inhibitory small molecule or a PD-1 ligand mimetic.

13. The composition of claim 11, wherein said PD-1 inhibitor is an anti-PD-1 monoclonal antibody.

14. The composition of claim 11, wherein said PD-1 inhibitor is an anti-Programmed death-ligand 1 (PD-L1) monoclonal antibody.

15. The composition of claim 1, wherein said composition reduces tumor development, tumor growth, tumor progression, metastatic spread to a different site or a combination thereof.

16. The composition of claim 1, wherein said composition stimulates an endogenous immune system.

17. The composition of claim 1, wherein said composition stimulates generation of an ASPH-specific B cell immune response, generation of an ASPH-specific T cell immune response, or generation of a combination thereof.

18. The composition of claim 1, wherein said composition stimulates activation of a cluster of differentiation 8 (CD8)⁺ cell, activation of a cluster of differentiation 4 (CD4)+ cell, or activation of a combination thereof.

19. The composition of claim 1, wherein said tumor is a cancer with low mutation burdens.

20. An immunotherapeutic method of treating a tumor or inhibiting tumor metastasis in a subject, comprising: administering said subject with a vaccine construct for an immunization against a purified tumor antigen, said tumor being characterized as comprising a low frequency of neoantigen expression, and administering an checkpoint inhibitor, wherein the method potentiates an anti-tumor immune response without inducing autoimmunity in said subject.

21. The method of claim 20, wherein said antigen is an ASPH or an antigen fragment thereof.

22. The method of claim 21, wherein said vaccine construct expresses a purified ASPH antigen.

23. The method of claim 22, wherein said purified ASPH antigen comprises a purified N-terminal ASPH peptide, wherein said purified ASPH antigen comprises a purified N-terminal ASPH peptide (SEQ ID NO: 47).

24. The method of claim 22, wherein said purified ASPH antigen comprises a purified C-terminal ASPH peptide, wherein said purified ASPH antigen comprises a purified C-terminal ASPH peptide (SEQ ID NO: 43).

25. The method of claim 22, wherein said purified ASPH antigen is a purified peptide selected from the group consisting of SEQ ID NOs: 1-45.

26. The method of claim 22, wherein said purified ASPH antigen comprises a HLA class II restricted sequence of TGYTELVKSLERNWKLI (SEQ ID NO: 11), or wherein said purified ASPH antigen comprises a HLA class I sequence YPQSPRARY (SEQ ID NO:26).

27. (canceled)

28. The method of claim 20, wherein said vaccine construct comprises a phage vaccine or a dendritic cell vaccine.

29. The method of claim 28, wherein said phage vaccine is a lambda phage-based vaccine or wherein said dendritic cell vaccine comprises an isolated ASPH-loaded dendritic cell.

30. The method of claim 20, wherein said checkpoint inhibitor is a PD-1 inhibitor.

31. The method of claim 30, wherein said PD-1 inhibitor is a PD-1 inhibitory antibody, a PD-1 inhibitory nucleic acid, a PD-1 inhibitory small molecule or a PD-1 ligand mimetic.

32. The method of claim 30, wherein said PD-1 inhibitor is an anti-PD-1 monoclonal antibody.

33. The method of claim 30, wherein said PD-1 inhibitor is an anti-PD-L1 monoclonal antibody.

34. The method of claim 20, wherein said immunization comprises a prophylactic immunization and a booster immunization.

35. The method of claim 34, wherein said prophylactic immunization comprises administering said vaccine construct to said subject three times spaced one week apart.

36. The method of claim 34, wherein said booster immunization comprises administering said vaccine construct to said subject three times spaced one week apart.

37. The method of claim 20, wherein said checkpoint inhibitor is administered concurrently and/or sequentially with said vaccine construct.

38. The method of claim 20, wherein said checkpoint inhibitor is administered twice per week for 5 or 6 weeks concurrently and/or sequentially with a vaccine.

39. The method of claim 20, wherein said tumor is a cancer with low mutation burden.

40. The method of claim 20, wherein said tumor is a solid tumor.

41. The method of claim 20, wherein said tumor is selected from hepatocellular carcinoma, cholangiocarcinoma, non-small cell lung cancer, breast cancer, triple negative breast cancer, gastric cancer, pancreatic cancer, esophageal cancer, soft tissue cancer, sarcoma, osteosarcoma, colon cancer, renal cancer, myeloid leukemia, prostate cancer, glioblastoma and lymphoid leukemia.

42. The method of claim 41, wherein said tumor is hepatocellular carcinoma.

43. The method of claim 20, wherein said method is associated with reducing tumor development, tumor growth, tumor progression, metastatic spread to a different site, or a combination thereof.

44. The method of claim 20, wherein said method is associated with stimulating an endogenous immune system.

45. The method of claim 20, wherein said method is associated with generation of an ASPH-specific B cell immune response, generation of an ASPH-specific T cell immune response, or generation of a combination thereof.

46. The method of claim 20, wherein said method is associated with activation of a CD8+ cell, activation of a CD4+ cell, or activation of a combination thereof.

47. (canceled)

48. A combinatorial composition comprising a vaccine construct for an immunization against a purified tumor antigen, said tumor being characterized as comprising a low frequency of neoantigen expression, and a checkpoint inhibitor.

49. The composition of claim 48, wherein said antigen is an ASPH or an antigen fragment thereof.

50. The composition of claim 49, wherein said vaccine construct expresses a purified ASPH antigen.

51. The composition of claim 50, wherein said purified ASPH antigen comprises a purified N-terminal ASPH peptide, or wherein said purified ASPH antigen comprises a purified C terminal ASPH peptide.

52. (canceled)

53. The composition of claim 50, wherein said purified ASPH antigen is a purified peptide selected from the group consisting of SEQ ID NOs: 1-45.

54. The composition of claim 50, wherein said purified ASPH antigen comprises a human leukocyte antigen (HLA) class II restricted sequence of TGYTELVKSLERNWKLI (SEQ ID NO: 11), or wherein said purified ASPH antigen comprises a HLA class I restricted sequence of YPQSPRARY (SEQ ID NO:26).

55. (canceled)

56. The composition of claim 48, wherein said vaccine construct comprises a phage vaccine or a dendritic cell vaccine.

57. The composition of claim 56, wherein said phage vaccine is a lambda phage-based vaccine or wherein said dendritic cell vaccine comprises an isolated ASPH-loaded dendritic cell.

58. The composition of claim 48, wherein said checkpoint inhibitor is a PD-1 inhibitor.

59. The composition of claim 58, wherein said PD-1 inhibitor is a PD-1 inhibitory antibody, a PD-1 inhibitory nucleic acid, a PD-1 inhibitory small molecule or a PD-1 ligand mimetic.

60. The composition of claim 58, wherein said PD-1 inhibitor is an anti-PD-1 monoclonal antibody.

61. The composition of claim 58, wherein said PD-1 inhibitor is an anti-PD-L1 monoclonal antibody.

62. An immunotherapeutic method for inhibiting metastasis in a subject, comprising: concurrently and/or sequentially administering to said subject a vaccine construct for an immunization against a purified tumor antigen and an immune checkpoint inhibitor.

63. The method of claim 20, wherein the vaccine is administered through intradermal, subcutaneous, intranasal, intramuscular, intratumoral, intranodal, intralymphatic, intravenous, intragastric, intraperitoneal, intravaginal, intravesical, or percutaneous routes.

64. (canceled)

65. (canceled)

66. The composition of claim 19, wherein the low mutation burden comprises 0.001 to ≤1 somatic mutation/megabase.

67. (canceled)

68. (canceled)

69. The method of claim 20, wherein the tumor is a primary tumor and the method comprises concurrently and/or sequentially administering to said subject a vaccine construct for immunization against a purified tumor antigen and an immune modulator.
